(19) United States
(12) Patent Application Publication
(10) Pub. No.: US 2025/0073328 A1
(43) Pub. Date: Mar. 6, 2025

(54) COMPOSITIONS AND METHODS FOR TREATING OR PREVENTING HIV INFECTION

(71) Applicants: The United States Government As Represented By The Department Of Veterans Affairs, Washington, DC (US); Icahn School of Medicine at Mount Sinai, New York, NY (US); Molsoft, San Diego, CA (US); The Scripps Research Institute, La Jolla, CA (US)

(72) Inventors: Catarina E. Hioe, New York, NY (US); Maxim Totrov; Jiang Zhu, La Jolla, CA (US)

(21) Appl. No.: 18/821,631

(22) Filed: Aug. 30, 2024

Related U.S. Application Data
(60) Provisional application No. 63/580,015, filed on Sep. 1, 2023.

(51) Int. Cl. A61K 39/21; A61K 39/00; A61P 31/18; C07K 14/005

(52) U.S. Cl. CPC A61K 39/21; A61P 31/18; C07K 14/005; A61K 2039/575; A61K 2039/645; C12N 2740/16122; C12N 2740/16134; C12N 2740/16171

(57) ABSTRACT

Disclosed are trimeric complexes comprising an uncleaved prefusion optimized gp140 env trimer. Disclosed are compositions comprising a trimeric complex, wherein the trimeric complex comprises an uncleaved prefusion optimized gp140 env trimer. Disclosed are methods of inducing an immune response against HIV in a subject comprising administering one or more of the disclosed compositions to a subject in need thereof. Disclosed are methods of generating neutralizing antibodies (nAbs) to HIV in a subject comprising administering one or more of the disclosed compositions to a subject in need thereof. Disclosed are method of treating a subject infected with HIV comprising administering one or more of the disclosed compositions to a subject in need thereof. Disclosed are methods of inducing an immune response against HIV in a subject comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct, wherein the nucleic acid construct comprises a nucleic acid sequence that encodes for a polypeptide comprising an HIV-1 derived V1V2 domain and a trimer-forming scaffold.

### CROSS-REFERENCE TO RELATED APPLICATIONS

This claims the benefit of U.S. Provisional Patent Application No. 63/580,015, filed Sep. 1, 2023, which is incorporated by reference herein in its entirety.

### STATEMENT REGARDING FEDERALLY SPONSORED RESEARCH

This invention was made with government support under AI148327, AI139290, AI1456559, HHSN272201800004C, AI140909, and T23 AI07647 awarded by National Institute of Health; and I01BX005616 and 1IK6BX004607 awarded by the US Department of Veterans Affairs. The government has certain rights in the invention.

### REFERENCE TO SEQUENCE LISTING

The Sequence Listing submitted Nov. 19, 2024 as a xml file named “37759.0601U2.xml,” created on Nov. 19, 2024, and having a size of 15,614 bytes is hereby incorporated by reference pursuant to 37 C.F.R. § 1.52(e)(5).

### BACKGROUND

Developing HIV envelope (Env) immunogens capable of eliciting antibodies (Abs) effective against a broad array of HIV-1 isolates is a major challenge in HIV vaccine development. Phase 2b/3 vaccine trials, including the recent HVTN 706, HVTN 705, and HVTN 702 trials testing different vaccine platforms and regimens to elicit cross-reactive Abs against Env, have yielded no efficacy signals. HIV-1 vaccine candidates designed to generate broadly neutralizing antibodies (bNAbs) have not attained their ultimate goals, although a germline-targeting strategy utilizing a self-assembling nanoparticle vaccine with 60 copies of gp120 engineered outer domain (eOD-GT8 60mer) was reported to stimulate precursors of VRC01-class bNAbs against the CD4-binding site (CD4bs) in nearly all vaccine recipients in a phase I trial. During HIV-1 infection, bNAbs are produced after multiple years of chronic infection only in a small subset of HIV-1-seropositive individuals. While exposure to diverse variants over years has been implicated in promoting or guiding bNAb development and maturation, other factors contributing to the generation of bNAbs are not fully understood.

### BRIEF SUMMARY

Disclosed are trimeric complexes comprising an uncleaved prefusion optimized gp140 env trimer.

Disclosed are trimeric complexes comprising three monomeric polypeptides, wherein each monomeric polypeptide comprises an uncleaved prefusion optimized gp140 env.

Disclosed are polypeptides comprising an uncleaved prefusion optimized gp140 env.

Disclosed are nucleic acid sequences capable of encoding a monomeric polypeptide, wherein the monomeric polypeptide comprises a gp120 subunit and a gp41 subunit.

Disclosed are nucleic acid constructs comprising a nucleic acid sequence capable of encoding a monomeric polypeptide, wherein the monomeric polypeptide comprises a gp120 subunit and a gp41 subunit.

Disclosed are compositions comprising a trimeric complex, wherein the trimeric complex comprises an uncleaved prefusion optimized gp140 env trimer.

Disclosed are compositions comprising a trimeric complex comprising three monomeric polypeptides, wherein each monomeric polypeptide comprises an uncleaved prefusion optimized gp140 env.

Disclosed are compositions comprising a nucleic acid sequence capable of encoding a monomeric polypeptide, wherein the monomer polypeptide comprises a gp120 subunit and a gp41 subunit.

Disclosed are compositions comprising a nucleic acid construct comprising a nucleic acid sequence capable of encoding a monomeric polypeptide, wherein the monomeric polypeptide comprises a gp120 subunit and a gp41 subunit.

Disclosed are methods of inducing an immune response against HIV in a subject comprising administering one or more of the disclosed compositions to a subject in need thereof.

Disclosed are methods of generating neutralizing antibodies (nAbs) to HIV in a subject comprising administering one or more of the disclosed compositions to a subject in need thereof.

Disclosed are methods of generating antibodies that are cross-reactive against a broad array of HIV isolates comprising administering one or more of the disclosed compositions to a subject in need thereof.

Disclosed are method of treating a subject infected with HIV comprising administering one or more of the disclosed compositions to a subject in need thereof.

Disclosed are method of preventing HIV-1 infection in a subject comprising administering one or more of the disclosed compositions to the subject.

Disclosed are methods of immunizing a subject against HIV comprising administering one or more of the disclosed compositions to a subject in need thereof.

Disclosed are methods of inducing an immune response against HIV in a subject comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct, wherein the nucleic acid construct comprises a nucleic acid sequence that encodes for a polypeptide comprising an HIV-1 derived V1V2 domain and a trimer-forming scaffold.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1B show an antigenicity profile of V1V2-2J9C. FIG. 1A) Culture supernatant from 293T cells transfected with the A244 V1V2-2J9C-encoding plasmid (418H) was tested using Octet biolayer interferometry (BLI) for reactivity with PG9 and 2158. Each mAb (5 μg/ml) was applied onto anti-human IgG Fc (AHC) biosensors and reacted with titrated amounts of V1V2-2J9C-containing supernatant. The V1V2-2J9C binding was measured over time. FIG. 1B) ELISA reactivity of A244 V1V2-219C with mAbs against different Env regions. The V1V2-2J9C protein (2 μg/ml) was coated onto ELISA wells and reacted with mAbs (10 μg/ml each) specific for the CD4bs, V1V2 (V2i, V2p, V2q), V3, C5, or irrelevant parvovirus antigen.

FIGS. 2A-2D show the antigenicity of different A244 UFO-BG Env and V1V2 constructs. FIG. 2A) Reactivity of UFO-BG.WT vs UFO-BG.ΔV3 with mAbs against different Env regions to verify the loss of anti-V3 mAb reactivity with UFO-BG.ΔV3. FIG. 2B) Enhanced PG9 reactivity against UFO-BG.WT in complex with 2158 but not the other V2i mAbs. FIG. 2C) Enhanced PG9 reactivity similarly observed with UFO-BG.ΔV3 in complex with 2158. FIG. 2D) Enhanced PG9 reactivity also detected against V1V2-2J9C in complex with 2158. ELISA was performed by coating wells with antigens at 2 μg/ml or titrating amounts followed by incubation with the designated mAbs. In panel A, mAb binding was detected with an alkaline phosphatase (AP)-coupled secondary anti-human IgG antibody. For panels B-D, the different antigens were pre-incubated with each mAb at a 1:3 ratio. Uncomplexed antigens were treated an irrelevant anti-parvovirus mAb 1418 for controls. Respective antigens pre-treated with PG9 were tested in parallel. The antigen and mAb mixtures were diluted serially, coated onto wells, and reacted with biotinylated PG9, followed with AP-streptavidin and luminescent substrate.

FIGS. 3A-3C shows an immunization of rabbits with DNA and protein vaccines to elicit Abs responses to V1V2. FIG. 3A) Rabbits (n=5/group) received V1V2-2J9C-encoding DNA vaccine by gene gun and a protein vaccine made of UFO-BG.ΔV3 or V1V2-2J9C, either uncomplexed (UC) or as immune complex (IC) with V2i mAb 2158. Each rabbit was immunized four times at one-month intervals and blood was collected two weeks after each vaccine dose. FIG. 3B) Induction of Ab responses against the homologous UFO-BG.ΔV3 or V1V2-2J9C antigens over time in Group 1A (DNA/UFO-UC) and Group 1B (DNA/UFO-IC).

FIG. 3C) Induction of Ab responses over time against V1V2-2J9C, but not UFO-BG.ΔV3, in Group 2A (DNA/V1V2-UC) and Group 2B (DNA-V1V2-IC). FIGS. 4A-4C show the profiles of binding Abs induced in immunized rabbits from the different groups. (FIG. 4A, 4B) Relative levels of Abs binding to 15 different antigens in Groups 1A, B (A) and Groups 2A, B (B). Serially diluted serum samples from individual animals collected after the last vaccination were tested for IgG reactivity against 15 different antigens in the Luminex multiplex bead assays. Area under the titration curve (AUC) was calculated and subtracted by pre-bleed AUC to yield AAUC values. Median values and median fold changes of IC/UC were also calculated. (FIG. 4C, 4D) Differences in serum Ab levels elicited by ICs and their uncomplexed counterparts. AUC values from each of the five animals in Group 1B and Group 2B were subtracted by those of Group 1A and Group 2A, respectively. **p<0.01; p>0.05 was left unmarked.

FIG. 5 shows neutralization activities elicited in the four rabbit groups. Neutralization of HIV-I pseudoviruses from different tiers and clades was tested in a standard assay using the TZM.bl target cells. Serum samples collected two weeks after the last immunization (week 14) were evaluated for neutralization activity. Virus pseudotyped with MLV was used as negative control, and control mAbs were also included for comparison. Bold values denote ID50 titers measurable above cut-offs (above MLV control <30 or >3-fold higher than MLV control of >30).

FIGS. 6A and 6B show diminishing TH023.6 neutralization by serum treatment with UFO-BG.ΔV3 or by N160K mutation. Virus neutralization was performed as in FIG. 5. (FIG. 6A) Serially diluted serum was pre-incubated with UFO-BG.ΔV3, A244 V1V2-2J9C, or ZM109-cV2 peptide (each at a fixed amount of 50 μg/ml) before testing in the neutralization assay against TH023.6 virus. (FIG. 6B) Neutralizing activity of rabbit sera from Groups 1A and 1B against TH023.6 and Ce1176_A3 WT vs. the respective N160K mutants. ID50 titers above the MLV ID50 values are plotted.

FIGS. 7A-7B show V1V2-specific ADCP activities detected in sera from the four rabbit groups. (FIG. 7A) Serially diluted serum was tested for ADCP with A244 V1V2-2J9C-coated beads and THP-1 phagocytes. Sera from immunized rabbits were tested in parallel with a pre-bleed serum pool. Titration curves were plotted for ADCP scores over serum dilutions. AUC values were then calculated and background-subtracted to yield AAUC. (FIG. 7B) ADCP potency for each rabbit serum was calculated as the ratios of ADCP/IgG against A244-V1V2-2J9C. *p<0.05; ns, p>0.05 by Mann-Whitney test. ns, not significant.

FIG. 8 shows an antigenicity profile of V1V2-2J9C. A DNA plasmid 418H encoding A244 V1V2-2J9C were evaluated using V2q mAb PG9 and V2i mAb 2158 (2 μg/ml each), followed by FITC-labeled goat anti-human IgG. Untreated COS-7 cells stained with antibodies served as control.

FIG. 9 shows enhanced PG9 reactivity with different V1V2-scaffolds in complex with 2158. ZM53 V1V2-2J9C or ZM233 V1V2-1FD6 were pre-incubated with 2158, PG9, or 1418 (irrelevant control) at a 1:3 ratio. The antigen-mAb mixtures were diluted serially, coated onto wells, and reacted with biotinylated PG9, followed with AP-streptavidin and luminescent.

FIG. 10 shows recognition of 15 antigens by anti-Env mAbs with defined epitope specificities. ELISA was performed to test antigen reactivity with mAbs specific for three distinct V1V2 epitopes (V2q or V2 apex, V2i, and V2p), the V3 crown epitopes, the C terminus (C5) of gp120, and an irrelevant parvovirus antigen (860). Based on the OD values above control, the presence of V2q, V2i, and/or V2p epitopes in each of the 15 antigens is denoted in FIGS. 4A and 4B.

FIGS. 11A-11B show in FIG. 11A a comparison of Ab responses to cyclic V2 (cV2) peptides elicited in Groups 1A vs 1B. Beads coated with each cV2 peptide were reacted with serially diluted sera after the last vaccination dose. AUCs were subtracted with pre-bleed AUCs to yield ΔAUC values. *, p<0.05; ns, p>0.05 by Mann Whitney test. FIG. 11B shows correlation of IgG binding data from the multiplex bead assays for Groups 1A and 1B. Multiple bead assay data were from FIG. 4. Correlation analysis was performed using the Spearman test. Correlation coefficients are color coded according to the red (−1)-to-blue (+1) scale, while p values were presented in each box.

FIGS. 12A-12C show elicitation of Abs capable of blocking PG9 and 2158 in the four rabbit groups. Beads coated with C.ZM109 V1V2-1FD6 or A244 V1V2-2J9C (both bearing V2q and V2i epitopes) were treated with serially diluted immune sera collected after the final vaccination or pre-bleed serum pool. Biotinylated PG9 or 2158 mAbs were subsequently added, and the reduced binding of the respective mAbs by immune sera was presented as ΔAUC (AUC of prebleed sera—AUC of immune sera). FIG. 12A) Relative levels of PG9-blocking Abs in immune sera binding to ZM109 V1V2-1FD6. FIG. 12B) Lack of 2158-blocking Abs in immune sera when tested with ZM109 V1V2-1FD6. FIG. 12C) Relative levels of 2158-blocking Abs in immune sera when tested with A244 V1V2-2J9C.

FIG. 13 shows N160K abrogated TH023.6 neutralization by V2-apex mAbs without affecting the other mAbs.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a polypeptide” includes a plurality of such polypeptides, reference to “the nucleic acid sequence” is a reference to one or more nucleic acid sequences and equivalents thereof known to those skilled in the art, and so forth.

The word “or” as used herein means any one member of a particular list and also includes any combination of members of that list.

As used herein, the term “therapeutically effective amount” means an amount of a therapeutic, prophylactic, and/or diagnostic agent (e.g., one or more of the compositions disclosed herein) that is sufficient, when administered to a subject suffering from or susceptible to an infection, disease, disorder, and/or condition, to treat, alleviate, ameliorate, relieve, alleviate symptoms of, prevent, delay onset of, inhibit progression of, reduce severity of, and/or reduce incidence of the infection, disease, disorder, and/or condition.

By “treat” is meant to administer a polypeptide, composition, nucleic acid sequence, nucleic acid construct, or vaccine of the invention to a subject, such as a human or other mammal (for example, an animal model), that has an increased susceptibility for developing an infection, disease, or disorder (e.g. HIV infection) in order to prevent or delay onset of the infection, disease or disorder, prevent or delay a worsening of the effects of the infection, disease or disorder, or to partially or fully reverse the effects of the infection, disease or disorder. In some aspects, treat can mean to ameliorate a symptom of a disease or disorder (e.g. inflammation, chronic pain or depression).

By “prevent” is meant to minimize the chance that a subject who has an increased susceptibility for developing an infection, disease, or disorder will actually develop the infection, disease, or disorder.

As used herein, the term “subject” or “patient” can be used interchangeably and refer to any organism to which a peptide or composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates, and the like.

As used herein, the terms “administering” and “administration” refer to any method of providing a disclosed polypeptide, composition, nucleic acid construct, or vaccine of the invention to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed exosome so as to treat a subject.

The terms “variant” and “mutant” are used interchangeably herein. As used herein, the term “mutant” refers to a modified nucleic acid or protein which displays the same characteristics when compared to a reference nucleic acid or protein sequence. A variant can be at least 65, 70, 75, 80, 85, 90, 95, or 99 percent homologous or identical to a reference sequence. In some aspects, a reference sequence can be a portion (e.g. a fragment) of a gp120 nucleic acid sequence or protein sequence (e.g. fragment of gp120). Variants can also include nucleotide sequences that are substantially similar to sequences of peptide disclosed herein. A “variant” can mean a difference in some way from the reference sequence other than just a simple deletion of an N- and/or C-terminal amino acid. Variants can also or alternatively include at least one substitution and/or at least one addition; there may also be at least one deletion. Alternatively or in addition, variants can comprise modifications, such as non-natural residues at one or more positions with respect to a reference nucleic acid or protein.

Substitutions, deletions, insertions or any combination thereof may be used to arrive at a final derivative or variant. Generally, these changes are done on a few residues to minimize the alteration of the molecule. However, larger changes may be tolerated in certain circumstances.

Generally, the amino acid or nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3, 4 amino acid residue changes as compared to the parent or reference sequence of the invention, and shares or improves biological function, specificity and/or activity of the parent sequence.

Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence.

The term “percent (%) homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res. 25:3389-3402, 1997.) A preferred alignment of selected sequences in order to determine “% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

“Polypeptide” as used herein refers to any peptide, oligopeptide, gene product, expression product, or protein. A polypeptide is comprised of consecutive amino acids. The term “polypeptide” encompasses naturally occurring or synthetic molecules.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Trimeric Complexes

HIV envelope glycoprotein gp160 is a trimeric complex with three monomeric polypeptides, wherein each monomeric polypeptide comprises an ectodomain, transmembrane domain and intracellular (cytoplasmic) domain. Upon removal of the transmembrane and intracellular domains, each monomeric polypeptide comprises the remaining ectodomain envelope glycoprotein referred to as gp140 and comprises two subunits: gp120 and gp41. In some aspects, the gp140 monomeric polypeptides can also form trimeric complexes as described herein. In some aspects, the HIV envelope glycoprotein gp160 can be HIV-1 envelope glycoprotein gp160. In some aspects, HIV and HIV-1 can be used interchangeably.

HIV-1 envelope glycoprotein gp140 can form trimers by complexing three monomers of gp140 env. Disclosed herein are trimeric complexes comprising gp140 env trimers. In some aspects, each monomer of gp140 env can be chimeric wherein a portion is derived from one HIV-1 strain and another portion is derived from a second HIV-1 strain. For example, gp140 env can comprise gp120 from A244 strain and gp41 from BG505 strain. In some aspects, different chimeric gp140 polypeptides can be produced. In some aspects, any strain of HIV can be used for the gp120 subunit or gp41 subunit. In some aspects, the BG505 strain is used for the gp41 subunit but the gp120 subunit can be from any strain. In some aspects, the gp120 subunit can be a chimera of two different strains. For example, the gp120 subunit can be a chimera of CM244 and A244 strains.

Disclosed are trimeric complexes comprising an uncleaved prefusion optimized gp140 env trimer. Disclosed are trimeric complexes comprising three monomeric polypeptides, wherein each monomeric polypeptide comprises an uncleaved prefusion optimized gp140 env.

In some aspects, the uncleaved prefusion optimized gp140 env trimer comprises three monomeric polypeptides, wherein each monomeric polypeptide comprises a gp120 subunit and a gp41 subunit. Thus, disclosed are trimeric complexes comprising three monomeric polypeptides, wherein each monomeric polypeptide is an uncleaved prefusion optimized gp140 env, wherein each uncleaved prefusion optimized gp140 env comprises a gp120 subunit and a gp41 subunit. In some aspects, the three monomeric polypeptides form a trimeric complex.

In some aspects, each of the three monomeric polypeptides of a trimeric complex comprises an amino acid sequence of MDAMKRGLCCVLLLCGAVFVSPSQEIHARFRRGARSDNLWVTVYYGVPVWKEADTTLFCA SDAKAHETEVHNVWATHACVPTDPNPOEIDLENVTENFNMWKNNMVEOMOEDVISLWDOS LKPCVKLTPPCVTLHCTNANLTKANLTNVNNRTNVSNIIGNITDEVRNCSFNMTTELRDKKO KVHALFYKLDIVPIEDNNDSSEYRLINCNTSVIKOPCPKISFDPIPIHYCTPAGYAILKCNDKNF NGTGPCKNVSSVOCTHGIKPVVSTOLLLNGSLAEEEIIIRSENLTNNAKTIIVHLNKSVVINCTR PSNNTRTSIGRFYYFGDIIGDIRKAYCEINGTEWNKALKOVTEKLKEHFNNKPIIFOPPSGGDLE ITMHHFNCRGEFFYCNTTRLFNNTCIANGTIEGCNGNITLPCKIKOIINMWOGAGOAMYAPPIS GTINCVSNITGILLTRDGGATNNTNNETFRPGGGNIKDNWRNELYKYKVVQIEPLGVAPTRCK RRVVE [symbol] GGGGSGGGGSAVGIGAVFLGFLGAAGSTMGAASMTLTVQARNLLSGNPDWLPD MTVWGIKQLQARVLAVEtYLRDQQLLGIWGCSGKLICCTNVPWNSSWSNRNLSEIWDN MTWLQWDKEISNYTQIIYGLLEESQNQQEKNEQDLLALD (SEQ ID NO: 1) or a variant thereof. The underlined sequence is a signal sequence from CM244. The double underlined sequence is the gp120 sequence which is a chimera of A244 and CM244 (the first 11 amino acids in the N-terminus are from CM244 and the remaining gp120 is from A244). The bold, italicized amino acids within the GP120 represent amino acid substitutions from a wild type sequence (SEQ ID NO:2). For example, TIGPGQV (SEQ ID NO:10) from the wild type gp120 subunit sequence seen in SEQ ID NO:2 is removed and instead, SEQ ID NO:1 has a GR sequence. The dotted underlined sequence represents a linker between the gp120 and gp41 that helps make the polypeptide be uncleaved. The bold sequence is gp41 of BG505. The signal sequence, MDAMKRGLCCVLLLCGAVFVSPSQEIHARFRRGAR can be cleaved during translation.

In some aspects, SEQ ID NO:1 can be with our without the signal sequence. In some aspects, when the signal sequence has been cleaved, each of the three monomeric polypeptides of a trimeric complex comprises an amino acid sequence of SDNLWVTVYYGVPVWKEADTTLFCASDAKAHETEVHNVWATHACVPTDPNPOEIDLENVT ENFNMWKNNMVEOMOEDVISLWDOSLKPCVKLTPPCVTLHCTNANLTKANLTNVNNRTNV SNIIGNITDEVRNCSFNMTTELRDKKOKVHALFYKLDIVPIEDNNDSSEYRLINCNTSVIKOPCP KISFDPIPIHYCTPAGYAILKCNDKNFNGTGPCKNVSSVOCTHGIKPVVSTOLLLNGSLAEEEIII RSENLTNNAKTIIVHLNKSVVINCTRPSNNTRTSIGRFYYFGDIIGDIRKAYCEINGTEWNKALK QVTEKLKEHFNNKPIIFOPPSGGDLEITMHHFNCRGEFFYCNTTRLFNNTCIANGTIEGCNGNIT LPCKIKOIINMWOGAGOAMYAPPISGTINCVSNITGILLTRDGGATNNTNNETFRPGGGNIKDN WRNELYKYKVVOIEPLGVAPTRCKRRVVE [symbol] GGGGSGGGGSAVGIGAVFLGFLGAAGSTMG AASMTLTVQARNLLSGNPDWLPDMTVWGIKQLQARVLAVERYLRDQQLLGIWGCSGK LICCTNVPWNSSWSNRNLSEIWDNMTWLQWDKEISNYTQIIYGLLEESQNQQEKNEQDL LALD (SEQ ID NO:3). SEQ ID NO:3 is SEQ ID NO:1 without the signal sequence. Thus, a variant of SEQ ID NO:1 can be SEQ ID NO:3. Any of the trimeric complexes disclosed throughout that refers to SEQ ID NO:1 can also be considered to referring to SEQ ID NO:3, which is a variant of SEQ ID NO:1.

In some aspects, a trimeric complex of three polypeptides comprising SEQ ID NO:1 or SEQ ID NO:3 can be referred to as UFO-BGΔV3 as it contains at least a portion of gp120 from the A244 strain and at least portion of a gp41 from the BG505 strain and part of the V3 region of gp120 is deleted compared to a monomeric polypeptide (e.g., SEQ ID NO:2) of UFO-BG WT. In some aspects, SEQ ID NO:1 can be compared to a wild type sequence as shown in SEQ ID NO:2

(SEQ ID NO: 2)
MDAMKRGLCCVLLLCGAVFVSPSQEIHARFRRGARSDNLWVTVYYGVPVWKEADTTL
FCASDAKAHETEVHNVWATHACVPTDPNPQEIDLENVTENFNMWKNNMVEQMQEDV
ISLWDQSLKPCVKLTPPCVTLHCTNANLTKANLTNVNNRTNVSNIIGNITDEVRNCSFN
MTTELRDKKQKVHALFYKLDIVPIEDNNDSSEYRLINCNTSVIKQPCPKISFDPIPIHYCTP
AGYAILKCNDKNFNGTGPCKNVSSVQCTHGIKPVVSTQLLLNGSLAEEEIIIRSENLTNN
AKTIIVHLNKSVVINCTRPSNNTRTSITIGPGQVFYRTGDIIGDIRKAYCEINGTEWNKAL
KQVTEKLKEHFNNKPIIFQPPSGGDLEITMHHFNCRGEFFYCNTTRLFNNTCIANGTIEGC
NGNITLPCKIKQIINMWQGAGQAMYAPPISGTINCVSNITGILLTRDGGATNNTNNETFR

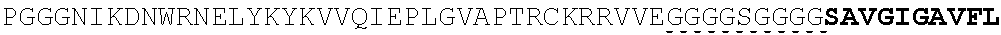

GFLGAAGSTMGAASMTLTVQARNLLSGNPDWLPDMTVWGIKQLQARVLAVERY
LRDQQLLGIWGCSGKLICCTNVPWNSSWSNRNLSEIWDNMTWLQWDKEISNYTQI
IYGLLEESQNQQEKNEQDLLALD.

The underlined sequence is a signal sequence from CM244. The double underlined sequence is the GP120 sequence which is a chimera of A244 and CM244 (the first 11 amino acids in the N-terminus are from CM244 and the remaining gp120 is from A244. The bold, italicized, double underlined sequence is a sequence that is replaced with GR as shown in the above SEQ ID NO:1. The dotted underlined sequence represents a linker between the GP120 and GP41 that helps make the polypeptide be uncleaved. The bold sequence is GP41 of BG505.

In some aspects, SEQ ID NO:2 can be with our without the signal sequence. In some aspects, when the signal sequence has been cleaved, each of the three monomeric polypeptides of a trimeric complex comprises an amino acid sequence of SDNLWVTVYYGVPVWKEADTTLFCASDAKAHETEVHNVWATHACVPTDPNPQEIDLENVT ENFNMWKNNMVEQMQEDVISLWDQSLKPCVKLTPPCVTLHCTNANLTKANLTNVNNRTNV SNIIGNITDEVRNCSFNMTTELRDKKQKVHALFYKLDIVPIEDNNDSSEYRLINCNTSVIKQPCP KISFDPIPIHYCTPAGYAILKCNDKNFNGTGPCKNVSSVQCTHGIKPVVSTQLLLNGSLAEEEIII RSENLTNNAKTIIVHLNKSVVINCTRPSNNTRTSITIGPGQVFYRTGDIIGDIRKAYCEINGTEW NKALKQVTEKLKEHFNNKPIIFQPPSGGDLEITMHHFNCRGEFFYCNTTRLFNNTCIANGTIEG CNGNITLPCKIKQIINMWQGAGQAMYAPPISGTINCVSNITGILLTRDGGATNNTNNETFRPGG GNIKDNWRNELYKYKVVQIEPLGVAPTRCKRRVVE [symbol] SAVGIGAVFLGFLGAA GSTMGAASMTLTVQARNLLSGNPDWLPDMTVWGIKQLQARVLAVERYLRDQQLLGIW GCSGKLICCTNVPWNSSWSNRNLSEIWDNMTWLQWDKEISNYTQIIYGLLEESQNQQEK NEQDLLALD (SEQ ID NO:4). SEQ ID NO:4 is SEQ ID NO:2 without the signal sequence. Thus, a variant of SEQ ID NO:2 can be SEQ ID NO:4. Any of the trimeric complexes disclosed throughout that refers to SEQ ID NO:2 can also be considered to referring to SEQ ID NO:4, which is a variant of SEQ ID NO:2.

In some aspects, the UFO-BG.ΔV3 is a trimeric complex of three gp140 polypeptides that comprise an A244 gp120 subunit, a BG505 gp41 ectodomain, and an optimized helical region 1 (of gp41) to form an uncleaved chimeric Env trimer.

In some aspects, a trimeric complex of three polypeptides each comprising SEQ ID NO:2 or SEQ ID NO:4 can be referred to as A244 UFO-BG (or UFO-BG WT) as it contains at least a portion of gp120 from the A244 strain and at least portion of a gp41 from the BG505 strain.

In some aspects, the gp120 subunit of an uncleaved prefusion optimized gp140 env monomeric polypeptide comprises a V3 truncation of amino acid sequence TIGPGQV (SEQ ID NO:10) (shown in SEQ ID NO:2), which is replaced with GR. Thus, in some aspects, the sequence TIGPGQV (SEQ ID NO:10) of SEQ ID NO:2 is deleted from the gp120 subunit of an uncleaved prefusion optimized gp140 env monomeric polypeptide and replaced with the sequence GR.

In some aspects, the gp120 subunit of the uncleaved prefusion optimized gp140 env monomeric polypeptide is from HIV-1 strain CRF01_ΔE A244 or is a chimera of A244 and another strain, such as CM244. In some aspects, the gp120 subunit comprises an amino acid sequence of SDNLWVTVYYGVPVWKEADTTLFCASDAKAHETEVHNVWATHACVPTDPNPQEIDLE NVTENFNMWKNNMVEQMQEDVISLWDQSLKPCVKLTPPCVTLHCTNANLTKANLTN VNNRTNVSNIIGNITDEVRNCSFNMTTELRDKKQKVHALFYKLDIVPIEDNNDSSEYRLI NCNTSVIKQPCPKISFDPIPIHYCTPAGYAILKCNDKNFNGTGPCKNVSSVQCTHGIKPVV STQLLLNGSLAEEEIIIRSENLTNNAKTIIVHLNKSVVINCTRPSNNTRTSITIGPGQVFYRT GDIIGDIRKAYCEINGTEWNKALKQVTEKLKEHFNNKPIIFQPPSGGDLEITMHHFNCRG EFFYCNTTRLFNNTCIANGTIEGCNGNITLPCKIKQIINMWQGAGQAMYAPPISGTINCVS NITGILLTRDGGATNNTNNETFRPGGGNIKDNWRNELYKYKVVQIEPLGVAPTRCKRRV VE (SEQ ID NO:5) or a variant thereof. In some aspects, the gp120 subunit comprises an amino acid sequence at least 70, 75, 80, 85, 90, 95, or 99% SEQ ID NO:5. In some aspects, the sequence TIGPGQV (SEQ ID NO:10) (as shown in italics above) can be replaced with GR which is shown in SEQ ID NO:6. In some aspects, the gp120 subunit comprises an amino acid sequence of SDNLWVTVYYGVPVWKEADTTLFCASDAKAHETEVHNVWATHACVPTDPNPQEIDLE NVTENFNMWKNNMVEQMQEDVISLWDQSLKPCVKLTPPCVTLHCTNANLTKANLTN VNNRTNVSNIIGNITDEVRNCSFNMTTELRDKKQKVHALFYKLDIVPIEDNNDSSEYRLI NCNTSVIKQPCPKISFDPIPIHYCTPAGYAILKCNDKNFNGTGPCKNVSSVQCTHGIKPVV STQLLLNGSLAEEEIIIRSENLTNNAKTIIVHLNKSVVINCTRPSNNTRTSIGRFYYFGDIIG DIRKAYCEINGTEWNKALKQVTEKLKEHFNNKPIIFQPPSGGDLEITMHHFNCRGEFFYC NTTRLFNNTCIANGTIEGCNGNITLPCKIKQIINMWQGAGQAMYAPPISGTINCVSNITGI LLTRDGGATNNTNNETFRPGGGNIKDNWRNELYKYKVVQIEPLGVAPTRCKRRVVE (SEQ ID NO:6). In some aspects, the gp120 subunit comprises an amino acid sequence at least 70, 75, 80, 85, 90, 95, or 99% identical to SEQ ID NO:6.

In some aspects, the gp41 subunit of the uncleaved prefusion optimized gp140 env monomeric polypeptide is from HIV-1 strain BG505. In some aspects, the gp41 subunit comprises the amino acid sequence SAVGIGAVFLGFLGAAGSTMGAASMTLTVQARNLLSGNPDWLPDMTVWGIKQLQARV LAVERYLRDQQLLGIWGCSGKLICCTNVPWNSSWSNRNLSEIWDNMTWLQWDKEISN YTQIlYGLLEESQNQQEKNEQDLLALD (SEQ ID NO:7). In some aspects, the gp41 subunit comprises an amino acid sequence at least 70, 75, 80, 85, 90, 95, or 99% identical to SEQ ID NO:7. In some aspects, the gp41 subunit is ectodomain gp41, meaning the transmembrane domain and cytoplasmic domains are not present. In some aspects, the gp41 ectodomain of BG505 comprises an optimized helical region 1. In some aspects, an optimized helical region 1 is described in He et al. HIV-1 Vaccine Design Through Minimizing Envelope Metastability. Sci Adv 2018 November; 4(11), hereby incorporated by reference in its entirety for its teaching of an optimized helical region 1.

In some aspects, the gp120 subunit of the uncleaved prefusion optimized gp140 env monomeric polypeptide comprises one or more amino acid substitutions compared to wild type gp140 (e.g., SEQ ID NO:1 compared to SEQ ID NO:2 (wild type)). In some aspects, the gp120 subunit of the uncleaved prefusion optimized gp140 env monomeric polypeptide comprises a R319Y substitution, a T320F substitution, or both. In some aspects, the positions of 319 and 320 are based on the standard HXB2 number system.

In some aspects, the disclosed trimeric complexes are immunogenic.

### C. Polypeptides

Disclosed are polypeptides comprising an uncleaved prefusion optimized gp140 env. In some aspects, the disclosed polypeptides are found as monomeric polypeptides. In some aspects, the disclosed polypeptides are monomeric polypeptides that form trimeric complexes as disclosed herein. In some aspects, the disclosed polypeptides comprising an uncleaved prefusion optimized gp140 env can be referred to as monomeric polypeptides, wherein three of the monomeric polypeptides form a trimeric complex as disclosed herein.

In some aspects, the disclosed polypeptides can comprise an amino acid sequence comprising the sequence of MDAMKRGLCCVLLLCGAVFVSPSQEIHARFRRGARSDNLWVTVYYGVPVWKEADTTLFCA SDAKAHETEVHNVWATHACVPTDPNPOEIDLENVTENFNMWKNNMVEOMOEDVISLWDOS LKPCVKLTPPCVTLHCTNANLTKANLTNVNNRTNVSNIIGNITDEVRNCSFNMTTELRDKKO KVHALFYKLDIVPIEDNNDSSEYRLINCNTSVIKOPCPKISFDPIPIHYCTPAGYAILKCNDKNF NGTGPCKNVSSVOCTHGIKPVVSTOLLLNGSLAEEEIIIRSENLTNNAKTIIVHLNKSVVINCTR PSNNTRTSIGRFYYFGDIIGDIRKAYCEINGTEWNKALKOVTEKLKEHFNNKPIIFOPPSGGDLE ITMHHFNCRGEFFYCNTTRLFNNTCIANGTIEGCNGNITLPCKIKOIINMWOGAGOAMYAPPIS GTINCVSNITGILLTRDGGATNNTNNETFRPGGGNIKDNWRNELYKYKVVOIEPLGVAPTRCK RRVV [symbol] SAVGIGAVFLGFLGAAGSTMGAASMTLTVQARNLLSGNPDWLPD MTVWGIKQLQARVLAVERYLRDQQLLGIWGCSGKLICCTNVPWNSSWSNRNLSEIWDN MTWLQWDKEISNYTQIIYGLLEESQNQQEKNEQDLLALD (SEQ ID NO: 1) or a variant thereof. The underlined sequence is a signal sequence from CM244. The double underlined sequence is the gp120 sequence which is a chimera of A244 and CM244 (the first 11 amino acids in the N-terminus are from CM244 and the remaining gp120 is from A244). The bold, italicized amino acids within the GP120 represent amino acid substitutions from a wild type sequence (SEQ ID NO:2). For example, TIGPGQV (SEQ ID NO:10) from the wild type gp120 subunit sequence seen in SEQ ID NO:2 is removed and instead, SEQ ID NO:1 has a GR sequence. The dotted underlined sequence represents a linker between the gp120 and gp41 that helps make the polypeptide be uncleaved. The bold sequence is gp41 of BG505. The signal sequence, MDAMKRGLCCVLLLCGAVFVSPSQEIHARFRRGAR can be cleaved during translation.

In some aspects, SEQ ID NO:1 can be with our without the signal sequence. In some aspects, when the signal sequence has been cleaved, each of the three monomeric polypeptides of a trimeric complex comprises an amino acid sequence of SDNLWVTVYYGVPVWKEADTTLFCASDAKAHETEVHNVWATHACVPTDPNPOEIDLENVT ENFNMWKNNMVEOMOEDVISLWDOSLKPCVKLTPPCVTLHCTNANLTKANLTNVNNRTNV SNIIGNITDEVRNCSFNMTTELRDKKOKVHALFYKLDIVPIEDNNDSSEYRLINCNTSVIKOPCP KISFDPIPIHYCTPAGYAILKCNDKNFNGTGPCKNVSSVOCTHGIKPVVSTOLLLNGSLAEEEIII RSENLTNNAKTIIVHLNKSVVINCTRPSNNTRTSIGRFYYFGDIIGDIRKAYCEINGTEWNKALK OVTEKLKEHFNNKPIIFOPPSGGDLEITMHHFNCRGEFFYCNTTRLFNNTCIANGTIEGCNGNIT LPCKIKOIINMWOGAGOAMYAPPISGTINCVSNITGILLTRDGGATNNTNNETFRPGGGNIKDN WRNELYKYKVVOIEPLGVAPTRCKRRVVE [symbol] SAVGIGAVFLGFLGAAGSTMG AASMTLTVQARNLLSGNPDWLPDMTVWGIKQLQARVLAVERYLRDQQLLGIWGCSGK LICCTNVPWNSSWSNRNLSEIWDNMTWLQWDKEISNYTQIIYGLLEESQNQQEKNEQDL LALD (SEQ ID NO:3). SEQ ID NO:3 is SEQ ID NO:1 without the signal sequence. Thus, a variant of SEQ ID NO:1 can be SEQ ID NO: 3. Any of the monomeric polypeptides disclosed throughout that refers to SEQ ID NO:1 can also be considered to referring to SEQ ID NO:3, which is a variant of SEQ ID NO:1.

In some aspects, a trimeric complex of three polypeptides, each comprising the sequence of SEQ ID NO: 1, can be referred to as UFO-BGΔV3.

In some aspects, the disclosed polypeptides can comprise an amino acid sequence comprising the sequence of

(SEQ ID NO: 2)
MDAMKRGLCCVLLLCGAVFVSPSQEIHARFRRGARSDNLWVTVYYGVPVWKEADTTL
FCASDAKAHETEVHNVWATHACVPTDPNPQEIDLENVTENFNMWKNNMVEQMQEDV
ISLWDQSLKPCVKLTPPCVTLHCTNANLTKANLTNVNNRTNVSNIIGNITDEVRNCSFN
MTTELRDKKQKVHALFYKLDIVPIEDNNDSSEYRLINCNTSVIKQPCPKISFDPIPIHYCTP
AGYAILKCNDKNFNGTGPCKNVSSVQCTHGIKPVVSTQLLLNGSLAEEEIIIRSENLTNN
AKTIIVHLNKSVVINCTRPSNNTRTSITIGPGQVFYRTGDIIGDIRKAYCEINGTEWNKAL
KQVTEKLKEHFNNKPIIFQPPSGGDLEITMHHFNCRGEFFYCNTTRLFNNTCIANGTIEGC
NGNITLPCKIKQIINMWQGAGQAMYAPPISGTINCVSNITGILLTRDGGATNNTNNETFR

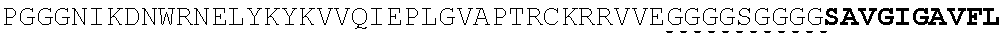

GFLGAAGSTMGAASMTLTVQARNLLSGNPDWLPDMTVWGIKQLQARVLAVERY
LRDQQLLGIWGCSGKLICCTNVPWNSSWSNRNLSEIWDNMTWLQWDKEISNYTQI
IYGLLEESQNQQEKNEQDLLALD.

The underlined sequence is a signal sequence from CM244. The double underlined sequence is the GP120 sequence which is a chimera of A244 and CM244 (the first 11 amino acids in the N-terminus are from CM244 and the remaining gp120 is from A244. The bold, italicized, double underlined sequence is a sequence that is replaced with GR in the above SEQ ID NO:1. The dotted underlined sequence represents a linker between the GP120 and GP41 that helps make the polypeptide be uncleaved. The bold sequence is GP41 of BG505.

In some aspects, a trimeric complex of three polypeptides, each comprising the sequence of SEQ ID NO:2, can be referred to as A244 UFO-BG or UFO-BG WT.

In some aspects, the gp120 subunit of an uncleaved prefusion optimized gp140 env monomeric polypeptide comprises a V3 truncation of amino acid sequence TIGPGQV (SEQ ID NO:10) (shown in SEQ ID NO:2), which is replaced with GR. Thus, in some aspects, the TIGPGQV (SEQ ID NO: 10) of SEQ ID NO:2 is deleted from the gp120 subunit of an uncleaved prefusion optimized gp140 env monomeric polypeptide and replaced with the sequence GR.

In some aspects, the gp120 subunit of the uncleaved prefusion optimized gp140 env monomeric polypeptide is from HIV-1 strain CRF01_AE A244 or is a chimera of A244 and another strain, such as CM244. In some aspects, the gp120 subunit comprises an amino acid sequence of SDNLWVTVYYGVPVWKEADTTLFCASDAKAHETEVHNVWATHACVPTDPNPQEIDLE NVTENFNMWKNNMVEQMQEDVISLWDQSLKPCVKLTPPCVTLHCTNANLTKANLTN VNNRTNVSNIIGNITDEVRNCSFNMTTELRDKKQKVHALFYKLDIVPIEDNNDSSEYRLI NCNTSVIKQPCPKISFDPIPIHYCTPAGYAILKCNDKNFNGTGPCKNVSSVQCTHGIKPVV STQLLLNGSLAEEEIIIRSENLTNNAKTIIVHLNKSVVINCTRPSNNTRTSITIGPGQVFYRT GDIIGDIRKAYCEINGTEWNKALKQVTEKLKEHFNNKPIIFQPPSGGDLEITMHHFNCRG EFFYCNTTRLFNNTCIANGTIEGCNGNITLPCKIKQIINMWQGAGQAMYAPPISGTINCVS NITGILLTRDGGATNNTNNETFRPGGGNIKDNWRNELYKYKVVQIEPLGVAPTRCKRRV VE (SEQ ID NO:5) or a variant thereof. In some aspects, the gp120 subunit comprises an amino acid sequence at least 70, 75, 80, 85, 90, 95, or 99% identical to SEQ ID NO:5. In some aspects, the sequence TIGPGQV (SEQ ID NO: 10) can be replaced with GR which is shown in SEQ ID NO:6. In some aspects, the gp120 subunit comprises an amino acid sequence of SDNLWVTVYYGVPVWKEADTTLFCASDAKAHETEVHNVWATHACVPTDPNPQEIDLE NVTENFNMWKNNMVEQMQEDVISLWDQSLKPCVKLTPPCVTLHCTNANLTKANLTN VNNRTNVSNIIGNITDEVRNCSFNMTTELRDKKQKVHALFYKLDIVPIEDNNDSSEYRLI NCNTSVIKQPCPKISFDPIPIHYCTPAGYAILKCNDKNFNGTGPCKNVSSVQCTHGIKPVV STQLLLNGSLAEEEIIIRSENLTNNAKTIIVHLNKSVVINCTRPSNNTRTSIGRFYYFGDIIG DIRKAYCEINGTEWNKALKQVTEKLKEHFNNKPIIFQPPSGGDLEITMHHFNCRGEFFYC NTTRLFNNTCIANGTIEGCNGNITLPCKIKQIINMWQGAGQAMYAPPISGTINCVSNITGI LLTRDGGATNNTNNETFRPGGGNIKDNWRNELYKYKVVQIEPLGVAPTRCKRRVVE (SEQ ID NO:6) or a variant thereof. In some aspects, the gp120 subunit comprises an amino acid sequence at least 70, 75, 80, 85, 90, 95, or 99% identical to SEQ ID NO:6.

In some aspects, the gp41 subunit of the uncleaved prefusion optimized gp140 env monomer is from HIV-1 strain BG505. In some aspects, the gp41 subunit comprises the amino acid sequence SAVGIGAVFLGFLGAAGSTMGAASMTLTVQARNLLSGNPDWLPDMTVWGIKQLQARV LAVERYLRDQQLLGIWGCSGKLICCTNVPWNSSWSNRNLSEIWDNMTWLQWDKEISN YTQIlYGLLEESQNQQEKNEQDLLALD (SEQ ID NO:7). In some aspects, the gp41 subunit comprises an amino acid sequence at least 70, 75, 80, 85, 90, 95, or 99% identical to SEQ ID NO:7. In some aspects, the gp41 subunit is ectodomain gp41, meaning the transmembrane domain and cytoplasmic domains are not present. In some aspects, the gp41 ectodomain of BG505 comprises an optimized helical region 1. In some aspects, an optimized helical region 1 is described in He et al. HIV-1 Vaccine Design Through Minimizing Envelope Metastability. Sci Adv 2018 November; 4(11), hereby incorporated by reference in its entirety for its teaching of an optimized helical region 1.

In some aspects, the gp120 subunit of the uncleaved prefusion optimized gp140 env monomeric polypeptide comprises one or more amino acid substitutions compared to wild type gp140 (e.g., SEQ ID NO:1 compared to SEQ ID NO:2). In some aspects, the gp120 subunit of the uncleaved prefusion optimized gp140 env monomeric polypeptide comprises a R319Y substitution, a T320F substitution, or both. In some aspects, the positions of 319 and 320 are based on the standard HXB2 number system.

In some aspects, the disclosed polypeptides are immunogenic in monomeric form or as trimeric complexes.

### D. Nucleic Acid Sequences and Constructs

As this specification discusses various polypeptide sequences, it is understood that the nucleic acid sequences that can encode monomeric polypeptides are disclosed and the nucleic acid constructs that comprise the nucleic acid sequences are also disclosed. This would include all degenerate sequences related to a specific polypeptide sequence, i.e. all nucleic acids having a sequence that encodes one particular polypeptide sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed polypeptide sequences. Any reference to a nucleic acid sequence capable of encoding can also be referred to as a nucleic acid sequence encoding. Thus, capable of encoding and encoding can be used interchangeably.

Disclosed are nucleic acid sequences capable of encoding any of the monomeric polypeptides disclosed herein. Further disclosed are nucleic acid constructs comprising the nucleic acid sequences capable of encoding any of the monomeric polypeptides disclosed herein.

Disclosed are nucleic acid sequences capable of encoding a monomeric polypeptide, wherein the monomeric polypeptide comprises a gp120 subunit and a gp41 subunit. Also disclosed are nucleic acid constructs comprising a nucleic acid sequence capable of encoding a monomeric polypeptide, wherein the monomeric polypeptide comprises a gp120 subunit and a gp41 subunit. Three of the monomeric polypeptides encoded by the disclosed nucleic acid sequences can complex together to form a trimeric complex thus resulting in the disclosed trimeric complexes comprising an uncleaved prefusion optimized gp140 env trimer. Disclosed are nucleic acid sequences capable of encoding a monomeric polypeptide, wherein the monomeric polypeptide comprises a gp120 subunit and a gp41 subunit, wherein the nucleic acid sequence comprises the sequence AAGCTTATGGATGCCATGAAGAGAGGACTGTGTTGCGTGCTGCTGCTCTGCGGCGC TGTGTTTGTCTCCCCTAGCCAAGAGATCCACGCTAGATTTAGAAGGGGAGCTAGAA GCGACAATCTGTGGGTGACAGTGTACTATGGCGTGCCCGTGTGGAAAGAGGCTGAC ACCACACTGTTCTGTGCCAGCGATGCCAAAGCTCACGAGACCGAGGTGCATAACGT CTGGGCTACCCATGCTTGCGTGCCCACAGATCCTAACCCTCAAGAGATCGATCTGGA GAACGTGACAGAGAACTTTAATATGTGGAAGAACAACATGGTGGAGCAGATGCAA GAGGACGTGATTAGCCTCTGGGACCAGTCTCTGAAGCCTTGCGTGAAACTCACACC CCCTTGTGTGACACTCCACTGCACAAACGCTAATCTGACCAAGGCCAATCTGACAA ACGTGAACAATAGAACAAATGTCTCCAATATTATTGGAAATATCACCGATGAGGTC AGAAATTGCAGCTTCAACATGACAACCGAGCTGAGAGATAAGAAGCAGAAGGTGC ACGCCCTCTTCTACAAACTGGACATCGTCCCCATCGAGGATAACAACGACTCCAGC GAGTATAGACTCATTAACTGCAACACCTCCGTGATCAAGCAGCCTTGCCCTAAGATC AGCTTCGACCCTATCCCCATCCACTATTGCACCCCCGCCGGCTACGCTATCCTCAAA TGCAACGACAAGAACTTCAATGGCACCGGCCCTTGCAAGAACGTGTCCAGCGTGCA GTGTACCCACGGAATCAAACCCGTGGTGTCCACCCAGCTGCTGCTGAATGGCTCTCT GGCTGAGGAAGAGATCATCATTAGATCCGAGAACCTCACCAACAATGCCAAGACCA TCATCGTGCATCTGAACAAATCCGTCGTGATCAACTGCACAAGACCCTCCAACAAC ACAAGGACCAGCATTGGAAGATTTTACTATTTTGGCGACATCATCGGCGATATTAG AAAGGCCTACTGCGAGATTAACGGCACCGAGTGGAATAAAGCTCTGAAACAAGTG ACAGAGAAGCTGAAAGAACATTTTAACAACAAGCCTATCATTTTCCAACCTCCCAG CGGCGGCGATCTCGAAATTACCATGCACCACTTTAATTGTAGGGGAGAGTTCTTCTA TTGTAATACCACCAGACTCTTCAACAACACATGCATCGCCAACGGCACCATCGAGG GCTGCAACGGCAACATTACACTGCCTTGCAAAATCAAGCAAATCATCAACATGTGG CAAGGCGCCGGCCAAGCCATGTATGCTCCCCCCATTTCCGGCACCATCAACTGCGTC AGCAATATTACCGGCATTCTGCTGACAAGGGATGGAGGCGCCACCAACAACACCAA CAACGAGACCTTTAGACCCGGCGGCGGCAACATTAAGGACAACTGGAGGAACGAG CTGTACAAGTATAAGGTCGTGCAGATTGAGCCCCTCGGAGTCGCCCCCACCAGATG CAAAAGGAGAGTCGTGGAAGGAGGAGGAGGCAGCGGAGGAGGCGGCAGCGCCGT GGGAATTGGAGCCGTGTTTCTCGGATTTCTGGGAGCTGCTGGCTCCACAATGGGAG CCGCCAGCATGACACTGACCGTGCAAGCTAGAAATCTGCTGAGCGGCAATCCCGAT TGGCTGCCCGACATGACCGTGTGGGGAATCAAGCAGCTGCAAGCTAGAGTGCTGGC CGTCGAAAGGTATCTGAGAGACCAGCAGCTGCTGGGAATCTGGGGCTGTAGCGGAA AGCTGATCTGTTGTACCAACGTGCCTTGGAACTCCAGCTGGAGCAATAGAAACCTCT CCGAGATCTGGGATAACATGACATGGCTGCAGTGGGACAAGGAGATTTCCAACTAC ACACAGATCATTTACGGACTGCTGGAGGAGTCCCAGAATCAGCAAGAGAAAAACG AGCAAGATCTGCTCGCTCTGGACGGATCC (SEQ ID NO:8) or a variant thereof. In some aspects, the bold sequences (which are restriction enzyme sites) can be absent in the optimized sequence. In some aspects, SEQ ID NO:8 encodes the amino acid sequence of SEQ ID NO:1

In some aspects, the nucleic acid construct is a non-viral or viral vector. In some aspects, non-viral vector is a plasmid.

The disclosed nucleic acid constructs can carry regulatory sequences that control the expression of the disclosed polypeptides in a host cell. It will be appreciated by those skilled in the art that the design of the vector, including the selection of regulatory sequences can depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods of expressing polypeptides in bacterial cells or fungal cells, e.g., yeast cells, are also well known in the art.

In some instances, the disclosed nucleic acid constructs further comprise a promoter operably linked to the nucleic acid sequence capable of encoding the disclosed polypeptides. In some instances, the promoter can be an inducible promoter. In some instances, the promoter can be a cell-specific promoter. The nucleic acid sequence capable of encoding the disclosed polypeptides can be functionally linked to a promoter. By “functionally linked” is meant such that the promoter can promote expression of the nucleic acid sequence, thus having appropriate orientation of the promoter relative to the nucleic acid sequence.

The terms “nucleic acid” and “polynucleotide” refer to RNA or DNA that is linear or branched, single-stranded or double-stranded or a hybrid thereof. The term also includes RNA/DNA hybrids. The following are non-limiting examples of polynucleotides: genes or gene fragments, exons, introns, mRNAs, tRNAs, rRNAs, ribozymes, cDNAs, recombinant polynucleotides, branched polynucleotides, plasmids, vectors, any sequence. Isolated DNA, isolated RNA of arbitrary sequence, nucleic acid probes and primers. Polynucleotides may include methylated nucleotides and nucleotide analogs, modified nucleotides such as uracil, other sugars and linking groups such as fluororibose and thiolate, as well as nucleotide branches. The nucleotide sequence may be further modified after polymerization, such as by conjugation with a labeling component. Other types of modifications included in this definition include caps, substitutions of one or more naturally occurring nucleotides with analogs, and polynucleotides as proteins, metal ions, labeling components, other polynucleotides or solids. It is the introduction of means for binding to a support. The polynucleotide can be obtained by chemical synthesis or may be of microbial origin.

### D. Vectors

In some aspects, the disclosed nucleic acid constructs are vectors. Thus, in some aspects, “nucleic acid construct” and “vector” can be used interchangeably. Disclosed are vectors comprising one or more of the nucleic acid sequences disclosed herein. Disclosed are vectors comprising a nucleic acid sequence capable of encoding a monomeric polypeptide, wherein the monomeric polypeptide comprises a gp120 subunit and a gp41 subunit.

In some aspects, the vectors can be used as a gene therapy tool. For example, the disclosed vectors can be delivered via a gene gun as naked plasmid DNA coated with gold particles.

In some aspects, the vector can be an expression vector. The term “expression vector” includes any vector, (e.g., a plasmid, cosmid or phage chromosome) containing a gene construct in a form suitable for expression by a cell (e.g., linked to a transcriptional control element). “Plasmid” and “vector” can be used interchangeably, as a plasmid is a commonly used form of vector. Moreover, the invention is intended to include other vectors which serve equivalent functions.

In some aspects, the vector can be a viral vector. For example, the viral vector can be an adeno-associated viral vector (AAV). In some aspects, the AAV can be AAV9. In some aspects, the vector can be a non-viral vector, such as a DNA based vector.

In some aspects, the vector is AAV9. In some aspects, a benefit of using AAV vectors can be that AAV is non-pathogenic in human and elicits a very mild immune response. In some aspects, the disclosed vectors are considered recombinant vectors. A recombinant AAV can lack two essential genes for viral integration and replication but remains primarily episomal and can persist in non-dividing cells for long periods of time. The tissue specificity of AAV can be determined by the viral capsid serotype, which allows targeting the gene of interest to specific tissues. In some aspects, AAV9 can be used to efficiently target the heart, although it also can target to other organs/tissues such as muscles, lungs, kidneys.

### 1. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence encoding a monomeric polypeptide comprising an amino acid sequence of MDAMKRGLCCVLLLCGAVFVSPSQEIHARFRRGARSDNLWVTVYYGVPVWKEADTTL FCASDAKAHETEVHNVWATHACVPTDPNPQEIDLENVTENFNMWKNNMVEQMQEDV ISLWDQSLKPCVKLTPPCVTLHCTNANLTKANLTNVNNRTNVSNIIGNITDEVRNCSFN MTTELRDKKQKVHALFYKLDIVPIEDNNDSSEYRLINCNTSVIKQPCPKISFDPIPIHYCTP AGYAILKCNDKNFNGTGPCKNVSSVQCTHGIKPVVSTQLLLNGSLAEEEIIIRSENLTNN AKTIIVHLNKSVVINCTRPSNNTRTSIGRFYYFGDIIGDIRKAYCEINGTEWNKALKQVTE KLKEHFNNKPIIFQPPSGGDLEITMHHFNCRGEFFYCNTTRLFNNTCIANGTIEGCNGNIT LPCKIKQIINMWQGAGQAMYAPPISGTINCVSNITGILLTRDGGATNNTNNETFRPGGGN IKDNWRNELYKYKVVQIEPLGVAPTRCKRRVVEGGGGSGGGGSAVGIGAVFLGFLGAA GSTMGAASMTLTVQARNLLSGNPDWLPDMTVWGIKQLQARVLAVERYLRDQQLLGI WGCSGKLICCTNVPWNSSWSNRNLSEIWDNMTWLQWDKEISNYTQIIYGLLEESQNQQ EKNEQDLLALD (SEQ ID NO:1). Also disclosed here are expression vectors comprising a nucleic acid sequence of SEQ ID NO: 8.

The “control elements” present in an expression vector are those non-translated regions of the vector—enhancers, promoters (e.g. a human pro-B-type natriuretic protein (hBNP) promoter), 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, u-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes B-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acid sequences, such as a nucleic acid sequence capable of encoding one or more of the disclosed polypeptides into a cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage

### E. Compositions

Disclosed are compositions comprising any of the disclosed trimeric complexes, monomeric polypeptides, nucleic acid sequences, nucleic acid constructs, or combinations thereof.

Disclosed are compositions comprising a trimeric complex, wherein the trimeric complex comprises an uncleaved prefusion optimized gp140 env trimer. Disclosed are compositions comprising a trimeric complex comprising three monomeric polypeptides, wherein each monomeric polypeptide comprises an uncleaved prefusion optimized gp140 env.

Disclosed are compositions comprising a nucleic acid sequence capable of encoding a monomeric polypeptide, wherein the monomer polypeptide comprises a gp120 subunit and a gp41 subunit. Disclosed are compositions comprising a nucleic acid construct comprising a nucleic acid sequence capable of encoding a monomeric polypeptide, wherein the monomeric polypeptide comprises a gp120 subunit and a gp41 subunit.

In some aspects, the composition is a vaccine. In some aspects, a vaccine can further comprise an adjuvant. In some aspects, any of the adjuvants described throughout can be present in the compositions.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In some aspects, a composition comprising a pharmaceutically acceptable carrier can be referred to as a pharmaceutical composition. Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed trimeric complexes, polypeptides, and/or nucleic acid constructs can be formulated and/or administered in or with a pharmaceutically acceptable carrier. As used herein, the term “pharmaceutically acceptable carrier” can refer to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed trimeric complexes, polypeptides, or nucleic acid constructs described herein, or a pharmaceutically acceptable salt or solvate thereof, and a pharmaceutically acceptable carrier, buffer, or diluent. In various aspects, the trimeric complex, polypeptide or nucleic acid construct of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the trimeric complexes, polypeptides, or nucleic acid constructs described herein and can also include a carrier such as a pharmaceutically acceptable carrier. For example, disclosed are pharmaceutical compositions, comprising the trimeric complexes, polypeptides, or nucleic acid constructs disclosed herein, and a pharmaceutically acceptable carrier. In one aspect, disclosed are pharmaceutical compositions comprising the disclosed trimeric complexes, polypeptides, or nucleic acid constructs. That is, a pharmaceutical composition can be provided comprising a therapeutically effective amount of at least one disclosed trimeric complexes, polypeptides, or nucleic acid constructs or at least one product of a disclosed method and a pharmaceutically acceptable carrier.

In certain aspects, the disclosed pharmaceutical compositions comprise the disclosed trimeric complexes, polypeptides, or nucleic acid constructs (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the peptides described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the trimeric complexes, polypeptides, or nucleic acid constructs of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

In order to enhance the solubility and/or the stability of the disclosed trimeric complexes, polypeptides, or nucleic acid constructs in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a disclosed trimeric complexes, polypeptides, or nucleic acid constructs (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### F. Vaccines

Disclosed are vaccines comprising one or more of the disclosed polypeptides, nucleic acid constructs or combinations thereof. For example, disclosed is a vaccine comprising an uncleaved prefusion optimized gp140 env trimer. Disclosed are trimeric complexes comprising three monomeric polypeptides, wherein each monomeric polypeptide comprises an uncleaved prefusion optimized gp140 env. Thus, in some aspects, the disclosed vaccines can be protein based, nucleic acid based, or a combination thereof.

In some aspects, a vaccine can comprise one or more of the disclosed compositions. In some aspects, a vaccine comprises one or more of the disclosed polypeptides, nucleic acid constructs, or compositions plus an adjuvant. In some aspects, an adjuvant can be any substance that enhances an immune response to the disclosed polypeptides, nucleic acid constructs, or compositions. In some aspects, the adjuvant can be an inorganic or organic adjuvant. In some aspects, an inorganic adjuvant can be an aluminum salt, such as, but not limited to, alum, aluminium hydroxide, aluminium phosphate, or combinations thereof. In some aspects, the organic adjuvant can be, but is not limited to, freund's complete or incomplete adjuvant, oil, squalene, unmethylated cytosine phosphoguanosine (CpG) oligonucleotides, plant extract QS-21, Monophosphoryl lipid A (MPL), dimethlydioctadecylammonium bromide (DDA), lipid nanoparticles (LNP), or combinations thereof. In some aspects, an adjuvant can be the combination of alum+CpG.

In some aspects, the disclosed vaccines can stimulate immunity to HIV. In some aspects, the disclosed vaccines can elicit a directed humoral and/or cellular immune response against one or more of the disclosed polypeptides, nucleic acid constructs, or compositions when administered to a host. In some aspects, the disclosed polypeptides can have substantially the same immunological activity as the full gp160 protein or as the full HIV. Thereby, the disclosed polypeptides comprise or consist of at least one epitope or antigenic determinant.

### G. Methods

The disclosed methods comprising administering one or more of the disclosed compositions to a subject in need thereof. In some aspects, any of the methods can comprise multiple administrations with the same composition or multiple administrations with multiple compositions.

In some aspects, the route of administration can vary among the different administrations. In some aspects, the route of administration is dependent on whether the composition is a protein based composition or a DNA based composition.

### 1. Method of Inducing an Immune Response

Disclosed are methods of inducing an immune response against HIV in a subject comprising administering one or more of the disclosed compositions to a subject in need thereof. In some aspects, the immune response is against HIV-1 and/or HIV-2.

In some aspects, the compositions comprise one or more of the disclosed trimeric complexes. For example, disclosed are methods of inducing an immune response against HIV in a subject comprising administering a composition or vaccine comprising a trimeric complex, wherein the trimeric complex comprises an uncleaved prefusion optimized gp140 env trimer to a subject in need thereof. Also disclosed methods of inducing an immune response against HIV in a subject comprising administering a composition or vaccine comprising a trimeric complex comprising three monomeric polypeptides, wherein each monomeric polypeptide comprises an uncleaved prefusion optimized gp140 env to a subject in need thereof.

In some aspects, the compositions comprise one or more of the disclosed nucleic acid constructs or vaccines. Thus, disclosed are methods of inducing an immune response against HIV in a subject comprising administering a composition or vaccine comprising a nucleic acid construct comprising a nucleic acid sequence capable of encoding a monomeric polypeptide, wherein the monomeric polypeptide comprises a gp120 subunit and a gp41 subunit.

An “immune response” or “immunological response” to a composition or vaccine is the development of a cellular and/or antibody-mediated immune response to the composition or vaccine of interest. Usually, an “immunological response” includes, but is not limited to, one or more of the following effects: an antibody specifically directed to one or more antigens (e.g., trimeric complexes, polypeptides) contained in or encoded by a composition or vaccine of interest. In some aspects, an immune response can lead to the production of B cells, helper T cells and/or cytotoxic T cells. In some aspects, a host can exhibit either a therapeutic or defensive immune response that enhances resistance to new infections and/or reduces the clinical severity of the infection/disease. Such protection is demonstrated by either the reduction or lack of symptoms normally exhibited by the infected host, the faster recovery period and/or the reduction in viral titer of the infected host.

In some aspects, the immune response is an antibody response. In some aspects, the antibody response is a neutralizing antibody response. In some aspects, the antibody response is an Fc effector function antibody response, which can include, but are not limited to antibody-dependent cellular cytotoxicity (ADCC) response, antibody-dependent cellular phagocytosis (ADCP) response, and complement-dependent cytotoxicity (CDC) response. In some aspects, the antibody response is an ADCP response. Thus, in some aspects, both neutralizing antibodies and Fc effector function antibodies can be generated in the disclosed methods.

In some aspects, the antibody response targets a broad array of HIV isolates, for example HIV-1 isolates.

In some aspects, the subject in need thereof is infected with HIV or at risk of becoming infected with HIV. For subjects at risk of becoming infected with HIV, inducing an immune response to HIV can prevent infection. For subjects infected with HIV, inducing an immune response to HIV can be therapeutic (e.g., prevent/reduce further infection/spread, eliminate/reduce virus, kill cells infected with virus).

In some aspects, methods of inducing an immune response against HIV in a subject comprises administering a combination of compositions to a subject in need thereof. For example, in some aspects, methods of inducing an immune response against HIV in a subject comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct comprising a nucleic acid sequence capable of encoding a monomeric polypeptide, wherein the monomeric polypeptide comprises a gp120 subunit and a gp41 subunit. In some aspects, the composition or vaccine comprising the trimeric complex and the composition or vaccine comprising the nucleic acid construct can be administering simultaneously or consecutively. In some aspects, administered simultaneously can mean administered within seconds or minutes of each other. In some aspects, administered consecutively can mean administered within hours, days, weeks, or months of each other. For example, in some aspects, administering a composition or vaccine comprising a nucleic acid construct comprising a nucleic acid sequence capable of encoding a monomeric polypeptide, wherein the monomeric polypeptide comprises a gp120 subunit and a gp41 subunit to a subject can be performed in order to prime the immune system. Then, days, weeks or months from the priming, a composition or vaccine comprising a trimeric complex, wherein the trimeric complex comprises an uncleaved prefusion optimized gp140 env trimer can be administered to the same subject to boost the immune system.

In some aspects, the composition or vaccine comprising the trimeric complex and the composition or vaccine comprising the nucleic acid construct can be administered separately. In some aspects, the composition or vaccine comprising the trimeric complex and the composition or vaccine comprising the nucleic acid construct can be administered as a single composition wherein both of the compositions are formulated together into one composition.

In some aspects, when administering two or more of the disclosed compositions or vaccines, the order of administration can be flexible and the timing of administration can be flexible.

In some aspects, the one or more compositions are administered subcutaneously, intramuscularly, orally, intravenously, or intradermally. In some aspects, when at least two compositions are administered to the subject, the compositions can be administered via the same route or via different routes.

### 2. Method of Generating Neutralizing Antibodies

Disclosed are methods of generating neutralizing antibodies (nAbs) to HIV in a subject comprising administering one or more of the disclosed compositions to a subject in need thereof. In some aspects, the nAbs are against HIV-1 and/or HIV-2.

In some aspects, the compositions comprise one or more of the disclosed trimeric complexes. Disclosed are methods of generating neutralizing antibodies to HIV in a subject comprising administering a composition or vaccine comprising a trimeric complex, wherein the trimeric complex comprises an uncleaved prefusion optimized gp140 env trimer to a subject in need thereof. Also disclosed methods of generating nAbs to HIV in a subject comprising administering a composition or vaccine comprising a trimeric complex comprising three monomeric polypeptides, wherein each monomeric polypeptide comprises an uncleaved prefusion optimized gp140 env to a subject in need thereof.

In some aspects, the compositions comprise one or more of the disclosed nucleic acid constructs or vaccines. Thus, disclosed are methods generating nAbs to HIV in a subject comprising administering a composition or vaccine comprising a nucleic acid construct comprising a nucleic acid sequence capable of encoding a monomeric polypeptide, wherein the monomeric polypeptide comprises a gp120 subunit and a gp41 subunit to a subject in need thereof.

In some aspects, nAbs can bind to the surface structures (antigens) of pathogens, such as viruses (e.g., HIV-1), bacteria, and microbial toxins, to prevent them from interacting with and infecting host cells. In some aspects, this neutralization process makes the pathogen (e.g., HIV-1) no longer infectious or pathogenic.

In some aspects, a “neutralizing antibody” can inhibit the entry of HIV-1 virus with a neutralization titer with ID50 of >25 and greater than the ID50 of control antibody. Broad and potent neutralizing antibodies can neutralize greater than about 50% of HIV-1 viruses tested (from diverse clades and different strains within a clade) in a neutralization assay. The neutralization ID50 titer of the plasma or serum antibody can be >25 to neutralize about 50% of the input virus in the neutralization assay.

In some aspects, the HIV is HIV-1 and the HIV-1 is strain TH023.6. Thus, in some aspects, the nAbs are generated to strain TH023.6. In some aspects, nAbs are generated to one or more of strains TH023.6, MN.3, MW965.26, CM244.EC1, Ce1176_A3, 96ZM651.1, Ce703010217_B6, and 246-F3_C10-2 that include tier 2 strains from one or more HIV-1 subtypes or circulating recombinant forms.

In some aspects, the subject in need thereof is infected with HIV or at risk of becoming infected with HIV. For subjects at risk of becoming infected with HIV, generating nAbs to HIV can prevent (neutralize) infection. For subjects infected with HIV, generating nAbs to HIV can be therapeutic (e.g., prevent/reduce further infection/spread, neutralize virus).

In some aspects, methods of generating nAbs to HIV in a subject comprises administering a combination of compositions to a subject in need thereof. For example, in some aspects, methods of generating nAbs to HIV in a subject comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct comprising a nucleic acid sequence capable of encoding a monomeric polypeptide, wherein the monomeric polypeptide comprises a gp120 subunit and a gp41 subunit. In some aspects, the composition or vaccine comprising the trimeric complex and the composition or vaccine comprising the nucleic acid construct can be administering simultaneously or consecutively. In some aspects, administered simultaneously can mean administered within seconds or minutes of each other. In some aspects, administered consecutively can mean administered within hours, days, weeks, or months of each other. For example, in some aspects, administering a composition or vaccine comprising a nucleic acid construct comprising a nucleic acid sequence capable of encoding a monomeric polypeptide, wherein the monomeric polypeptide comprises a gp120 subunit and a gp41 subunit to a subject can be performed in order to prime the immune system. Then, days, weeks or months from the priming, a composition or vaccine comprising a trimeric complex, wherein the trimeric complex comprises an uncleaved prefusion optimized gp140 env trimer can be administered to the same subject to boost the immune system.

In some aspects, the composition or vaccine comprising the trimeric complex and the composition or vaccine comprising the nucleic acid construct can be administered separately. In some aspects, the composition or vaccine comprising the trimeric complex and the composition or vaccine comprising the nucleic acid construct can be administered as a single composition wherein both of the compositions are formulated together into one composition.

In some aspects, when administering two or more of the disclosed compositions or vaccines, the order of administration can be flexible and the timing of administration can be flexible.

### 3. Method of Generating Cross-Reactive Antibodies

Disclosed are methods of generating antibodies that are cross-reactive against a broad array of HIV isolates comprising administering one or more of the disclosed compositions to a subject in need thereof. In some aspects, the HIV isolates are HIV-1 isolates and/or HIV-2 isolates.

In some aspects, the compositions comprise one or more of the disclosed trimeric complexes. For example, disclosed are methods of generating antibodies that are cross-reactive against a broad array of HIV-1 isolates comprising administering a composition or vaccine comprising a trimeric complex, wherein the trimeric complex comprises an uncleaved prefusion optimized gp140 env trimer to a subject in need thereof. Also disclosed are methods of generating antibodies that are cross-reactive against a broad array of HIV-1 isolates comprising administering a composition or vaccine comprising a trimeric complex comprising three monomeric polypeptides, wherein each monomeric polypeptide comprises an uncleaved prefusion optimized gp140 env.

In some aspects, the compositions comprise one or more of the disclosed nucleic acid constructs or vaccines. For example, disclosed are methods of generating antibodies that are cross-reactive against a broad array of HIV-1 isolates comprising administering a composition or vaccine comprising a nucleic acid construct comprising a nucleic acid sequence capable of encoding a monomeric polypeptide, wherein the monomeric polypeptide comprises a gp120 subunit and a gp41 subunit.

In some aspects, cross-reactive against a broad array of HIV-1 isolates can mean the antibodies are cross-reactive against heterologous strains from the same or different clade/circulating recombinant forms.

In some aspects, the subject in need thereof is infected with HIV or at risk of becoming infected with HIV. For subjects at risk of becoming infected with HIV, generating nAbs to HIV can prevent (neutralize) infection. For subjects infected with HIV, generating nAbs to HIV can be therapeutic (e.g., prevent/reduce further infection/spread, neutralize virus).

In some aspects, methods of generating antibodies that are cross-reactive against a broad array of HIV-1 isolates comprises administering a combination of compositions to a subject in need thereof. For example, in some aspects, methods of generating antibodies that are cross-reactive against a broad array of HIV-1 isolates comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct comprising a nucleic acid sequence capable of encoding a monomeric polypeptide, wherein the monomeric polypeptide comprises a gp120 subunit and a gp41 subunit. In some aspects, the composition or vaccine comprising the trimeric complex and the composition or vaccine comprising the nucleic acid construct can be administering simultaneously or consecutively. In some aspects, administered simultaneously can mean administered within seconds or minutes of each other. In some aspects, administered consecutively can mean administered within hours, days, weeks, or months of each other. For example, in some aspects, administering a composition or vaccine comprising a nucleic acid construct comprising a nucleic acid sequence capable of encoding a monomeric polypeptide, wherein the monomeric polypeptide comprises a gp120 subunit and a gp41 subunit to a subject can be performed in order to prime the immune system. Then, days, weeks or months from the priming, a composition or vaccine comprising a trimeric complex, wherein the trimeric complex comprises an uncleaved prefusion optimized gp140 env trimer can be administered to the same subject to boost the immune system.

In some aspects, the composition or vaccine comprising the trimeric complex and the composition or vaccine comprising the nucleic acid construct can be administered separately. In some aspects, the composition or vaccine comprising the trimeric complex and the composition or vaccine comprising the nucleic acid construct can be administered as a single composition wherein both of the compositions are formulated together into one composition.

In some aspects, when administering two or more of the disclosed compositions or vaccines, the order of administration can be flexible and the timing of administration can be flexible.

### 4. Methods of Treating

Disclosed are method of treating a subject infected with HIV comprising administering one or more of the disclosed compositions to a subject in need thereof. In some aspects, the HIV is HIV-1 and/or HIV-2.

In some aspects, the compositions comprise one or more of the disclosed trimeric complexes. For example, disclosed are methods of treating a subject infected with HIV-1 comprising administering a composition or vaccine comprising a trimeric complex, wherein the trimeric complex comprises an uncleaved prefusion optimized gp140 env trimer to a subject in need thereof. Also disclosed methods of treating a subject infected with HIV-1 comprising administering a composition or vaccine comprising a trimeric complex comprising three monomeric polypeptides, wherein each monomeric polypeptide comprises an uncleaved prefusion optimized gp140 env to a subject in need thereof.

In some aspects, the compositions comprise one or more of the disclosed nucleic acid constructs or vaccines. Thus, disclosed are methods of treating a subject infected with HIV-1 comprising administering a composition or vaccine comprising a nucleic acid construct comprising a nucleic acid sequence capable of encoding a monomeric polypeptide, wherein the monomeric polypeptide comprises a gp120 subunit and a gp41 subunit.

In some aspects, treating can refer to, by is not limited to, increase in immune response to HIV, a decrease in viral load, decrease in symptoms from HIV infection, decrease in developing AIDS, reduction or elimination of the standard antiretroviral therapy, and/or a reduction in the duration or frequency of the standard antiretroviral therapy.

In some aspects, the subject in need thereof is infected with HIV. For subjects infected with HIV, inducing an immune response to HIV can be therapeutic (e.g., prevent/reduce further infection/spread, eliminate/reduce virus, kill cells infected with virus).

In some aspects, methods of treating a subject infected with HIV comprises administering a combination of compositions to a subject in need thereof. For example, in some aspects, methods of treating a subject infected with HIV comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct comprising a nucleic acid sequence capable of encoding a monomeric polypeptide, wherein the monomeric polypeptide comprises a gp120 subunit and a gp41 subunit. In some aspects, the composition or vaccine comprising the trimeric complex and the composition or vaccine comprising the nucleic acid construct can be administering simultaneously or consecutively. In some aspects, administered simultaneously can mean administered within seconds or minutes of each other. In some aspects, administered consecutively can mean administered within hours, days, weeks, or months of each other. For example, in some aspects, administering a composition or vaccine comprising a nucleic acid construct comprising a nucleic acid sequence capable of encoding a monomeric polypeptide, wherein the monomeric polypeptide comprises a gp120 subunit and a gp41 subunit to a subject can be performed in order to prime the immune system. Then, days, weeks or months from the priming, a composition or vaccine comprising a trimeric complex, wherein the trimeric complex comprises an uncleaved prefusion optimized gp140 env trimer can be administered to the same subject to boost the immune system.

In some aspects, the composition or vaccine comprising the trimeric complex and the composition or vaccine comprising the nucleic acid construct can be administered separately. In some aspects, the composition or vaccine comprising the trimeric complex and the composition or vaccine comprising the nucleic acid construct can be administered as a single composition wherein both of the compositions are formulated together into one composition.

In some aspects, when administering two or more of the disclosed compositions or vaccines, the order of administration can be flexible and the timing of administration can be flexible.

In some aspects, the one or more compositions are administered subcutaneously, intramuscularly, orally, intravenously, or intradermally. In some aspects, when at least two compositions are administered to the subject, the compositions can be administered via the same route or via different routes

### 5. Methods of Preventing HIV Infection

Disclosed are method of preventing HIV-1 infection in a subject comprising administering one or more of the disclosed compositions to the subject. In some aspects, the HIV is HIV-1 and/or HIV-2.

In some aspects, the compositions comprise one or more of the disclosed trimeric complexes. For example, disclosed are methods of preventing HIV-1 infection in a subject comprising administering a composition or vaccine comprising a trimeric complex, wherein the trimeric complex comprises an uncleaved prefusion optimized gp140 env trimer to a subject in need thereof. Also disclosed methods of preventing HIV-1 infection in a subject comprising administering a composition or vaccine comprising a trimeric complex comprising three monomeric polypeptides, wherein each monomeric polypeptide comprises an uncleaved prefusion optimized gp140 env to a subject in need thereof.

In some aspects, the compositions comprise one or more of the disclosed nucleic acid constructs or vaccines. Thus, disclosed are methods of preventing HIV-1 infection in a subject comprising administering a composition or vaccine comprising a nucleic acid construct comprising a nucleic acid sequence capable of encoding a monomeric polypeptide, wherein the monomeric polypeptide comprises a gp120 subunit and a gp41 subunit.

In some aspects, preventing HIV-1 infection in a subject comprises neutralizing the virus prior to entry into cells, killing or lysing virus particles and virus-infected cells, and/or destroying virus particles or virus-infected cells with participation of effector cells.

In some aspects, methods of preventing HIV-1 infection in a subject comprises administering a combination of compositions to the subject. For example, in some aspects, methods of preventing HIV-1 infection in a subject comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct comprising a nucleic acid sequence capable of encoding a monomeric polypeptide, wherein the monomeric polypeptide comprises a gp120 subunit and a gp41 subunit. In some aspects, the composition or vaccine comprising the trimeric complex and the composition or vaccine comprising the nucleic acid construct can be administering simultaneously or consecutively. In some aspects, administered simultaneously can mean administered within seconds or minutes of each other. In some aspects, administered consecutively can mean administered within hours, days, weeks, or months of each other. For example, in some aspects, administering a composition or vaccine comprising a nucleic acid construct comprising a nucleic acid sequence capable of encoding a monomeric polypeptide, wherein the monomeric polypeptide comprises a gp120 subunit and a gp41 subunit to a subject can be performed in order to prime the immune system. Then, days, weeks or months from the priming, a composition or vaccine comprising a trimeric complex, wherein the trimeric complex comprises an uncleaved prefusion optimized gp140 env trimer can be administered to the same subject to boost the immune system.

In some aspects, the composition or vaccine comprising the trimeric complex and the composition or vaccine comprising the nucleic acid construct can be administered separately. In some aspects, the composition or vaccine comprising the trimeric complex and the composition or vaccine comprising the nucleic acid construct can be administered as a single composition wherein both of the compositions are formulated together into one composition.

In some aspects, when administering two or more of the disclosed compositions or vaccines, the order of administration can be flexible and the timing of administration can be flexible.

In some aspects, the one or more compositions are administered subcutaneously, intramuscularly, orally, intravenously, or intradermally. In some aspects, when at least two compositions are administered to the subject, the compositions can be administered via the same route or via different routes.

### 6. Methods of Immunizing/Vaccinizing

Disclosed are methods of immunizing a subject against HIV comprising administering one or more of the disclosed compositions to a subject in need thereof. In some aspects, the disclosed methods of immunizing can also be referred to as methods of vaccinating a subject in need thereof. In some aspects, the HIV is HIV-1 and/or HIV-2.

In some aspects, the compositions comprise one or more of the disclosed trimeric complexes. For example, disclosed are methods of immunizing a subject against HIV-1 comprising administering a composition or vaccine comprising a trimeric complex, wherein the trimeric complex comprises an uncleaved prefusion optimized gp140 env trimer to a subject in need thereof. Also disclosed methods of immunizing a subject against HIV-1 comprising administering a composition or vaccine comprising a trimeric complex comprising three monomeric polypeptides, wherein each monomeric polypeptide comprises an uncleaved prefusion optimized gp140 env to a subject in need thereof.

In some aspects, the compositions comprise one or more of the disclosed nucleic acid constructs or vaccines. Thus, disclosed are methods of immunizing a subject against HIV-1 comprising administering a composition or vaccine comprising a nucleic acid construct comprising a nucleic acid sequence capable of encoding a monomeric polypeptide, wherein the monomeric polypeptide comprises a gp120 subunit and a gp41 subunit.

In some aspects, the subject in need thereof is infected with HIV or at risk of becoming infected with HIV. For subjects at risk of becoming infected with HIV, inducing an immune response to HIV can prevent infection. For subjects infected with HIV, inducing an immune response to HIV can be therapeutic (e.g., prevent/reduce further infection/spread, eliminate/reduce virus, kill cells infected with virus).

In some aspects, immunizing results in the generation of an immune response to allow the subject to prevent infection with HIV or treat infection with HIV. In some aspects, the subject generates neutralizing antibodies. In some aspects, the subject generates a protective immune response effective to reduce or prevent infection. In some aspects, the subject generates antibodies effective against a broad array of HIV-1 isolates.

In some aspects, methods of immunizing a subject against HIV in a subject comprises administering a combination of compositions to a subject in need thereof. For example, in some aspects, methods of immunizing a subject against HIV comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct comprising a nucleic acid sequence capable of encoding a monomeric polypeptide, wherein the monomeric polypeptide comprises a gp120 subunit and a gp41 subunit. In some aspects, the composition or vaccine comprising the trimeric complex and the composition or vaccine comprising the nucleic acid construct can be administering simultaneously or consecutively. In some aspects, administered simultaneously can mean administered within seconds or minutes of each other. In some aspects, administered consecutively can mean administered within hours, days, weeks, or months of each other. For example, in some aspects, administering a composition or vaccine comprising a nucleic acid construct comprising a nucleic acid sequence capable of encoding a monomeric polypeptide, wherein the monomeric polypeptide comprises a gp120 subunit and a gp41 subunit to a subject can be performed in order to prime the immune system. Then, days, weeks or months from the priming, a composition or vaccine comprising a trimeric complex, wherein the trimeric complex comprises an uncleaved prefusion optimized gp140 env trimer can be administered to the same subject to boost the immune system.

In some aspects, the composition or vaccine comprising the trimeric complex and the composition or vaccine comprising the nucleic acid construct can be administered separately. In some aspects, the composition or vaccine comprising the trimeric complex and the composition or vaccine comprising the nucleic acid construct can be administered as a single composition wherein both of the compositions are formulated together into one composition.

In some aspects, when administering two or more of the disclosed compositions or vaccines, the order of administration can be flexible and the timing of administration can be flexible.

In some aspects, the one or more compositions are administered subcutaneously, intramuscularly, orally, intravenously, or intradermally. In some aspects, when at least two compositions are administered to the subject, the compositions can be administered via the same route or via different routes.

### H. Combination Therapy with a Second HIV Antigen, V1V2

In some aspects, any of the disclosed methods can be combined with administering a protein based vaccine or a nucleic acid based vaccine that can generate an immune response to V1V2. The protein based vaccine or nucleic acid based vaccine can be any of the V1V2 compositions described in U.S. Pat. No. 10,568,969, incorporated by reference in its entirety herein.

In some aspects, any of the disclosed methods can be combined with administering a trimeric complex comprising three polypeptides, wherein each polypeptide comprises an HIV-1 derived V1V2 domain and a trimer-forming scaffold. In some aspects, the trimer-forming scaffold is 2J9C. In some aspects, the scaffold can be any scaffold known to form trimers or other multimers thus helping in the formation of trimeric complexes (or multimeric complexes) of the disclosed polypeptides comprising a scaffold.

In some aspects, any of the disclosed methods can be combined with administering a nucleic acid construct, wherein the nucleic acid construct comprises a nucleic acid sequence that encodes for a polypeptide comprising an HIV-1 derived V1V2 domain and a trimer-forming scaffold.

In some aspects, a polypeptide comprising an HIV-1 derived V1V2 domain and a trimer-forming scaffold, 2J9C, comprises the amino acid sequence MDAMKRGLCCVLLLCGAVFVSPSQEIHARFRRGARSGSMKKVEAIIRPEKLEIVKKALS DAGYVGMTVSEVKGRGVQGGIVERYCVTLHCTNANLTKANLTNVNNRTNVSNIIGN ITDEVRNCSFNMTTELRDKKQKVHALFYKLDIVPIEDNNDSSEYRLINCREYIVDLIP KVKIELVVKEEDVDNVIDIICENARTGDPGDGKIFVIPVERVVRVRTKEEGKEALLE HGLEVLFOGPGHHHHHHHHSAWSHPOFEKEF (SEQ ID NO:9). The underlined sequence is a signal sequence from tPA that allows for secretion. The bold sequence is the V1V2 of A244. The bold underlined sequence is the 2J9C sequence. The italicized sequence is a His-tag that can be used for purification. The double underlined sequence is a strep tag that can also be used for purification.

In some aspects, any of the disclosed methods comprises administering a combination of compositions to a subject in need thereof. For example, in some aspects, methods of inducing an immune response against HIV in a subject comprise administering a composition or vaccine comprising a trimeric complex, wherein the trimeric complex comprises an uncleaved prefusion optimized gp140 env trimer, as disclosed herein, in combination with administering a composition or vaccine comprising a nucleic acid construct, wherein the nucleic acid construct comprises a nucleic acid sequence that encodes for a polypeptide comprising an HIV-1 derived V1V2 domain and a multimer-forming (e.g., trimer-forming) scaffold. In some aspects, the composition or vaccine comprising the trimeric complex and the composition or vaccine comprising the nucleic acid construct can be administering simultaneously or consecutively. In some aspects, administered simultaneously can mean administered within seconds or minutes of each other. In some aspects, administered consecutively can mean administered within hours, days, weeks, or months of each other. For example, in some aspects, administering a composition or vaccine comprising a nucleic acid construct can be performed in order to prime the immune system. Then, days, weeks or months from the priming, a composition or vaccine comprising a trimeric complex, wherein the trimeric complex comprises an uncleaved prefusion optimized gp140 env trimer can be administered to the same subject to boost the immune system.

In some aspects, the composition or vaccine comprising the trimeric complex and the composition or vaccine comprising the nucleic acid construct can be administered separately. In some aspects, the composition or vaccine comprising the trimeric complex and the composition or vaccine comprising the nucleic acid construct can be administered as a single composition wherein both of the compositions are formulated together into one composition.

In some aspects, when administering two or more of the disclosed compositions or vaccines, the order of administration can be flexible and the timing of administration can be flexible.

In some aspects, the one or more compositions are administered subcutaneously, intramuscularly, orally, intravenously, or intradermally. In some aspects, when at least two compositions are administered to the subject, the compositions can be administered via the same route or via different routes.

In one aspect of the disclosed methods, the compositions can be administered alone or in combination with one or more additional therapeutic agents. The additional therapeutic agents are selected based on the disease or symptom to be treated. A description of the various classes of suitable pharmacological agents and drugs may be found in Goodman and Gilman, The Pharmacological Basis of Therapeutics, (11th Ed., McGraw-Hill Publishing Co.) (2005).

### I. Dosages

Disclosed are dosing regimens comprising administering a single dose of one or more of the disclosed compositions to a subject in need thereof, wherein the single dose comprises an amount effective to generate an immune response.

Disclosed are dosing regimens comprising administering at least two doses of one or more of the disclosed compositions to a subject in need thereof, wherein each dose is the same concentration. In some aspects, each dose after a first dose can be decreased. In some aspects, each dose after a first dose can be increased.

In some aspects, a single dose can be a continuous administration. In some aspects, a continuous administration can be hours, days, weeks, or months. In some aspects, there can be two or more doses. In some aspects, the two or more doses can be administered days, weeks, or months apart.

### J. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for producing the disclosed compositions, the kit comprising polypeptides comprising an uncleaved prefusion optimized gp140 env or a nucleic acid sequence encoding polypeptides comprising an uncleaved prefusion optimized gp140 env. The kits also can contain vectors.

### EXAMPLES

### A. Example 1: Vaccination with Immune Complexes Modulates the Elicitation of Functional Antibodies Against HIV-1

### 1. Introduction

Developing HIV envelope (Env) immunogens capable of eliciting antibodies (Abs) effective against a broad array of HIV-1 isolates is a major challenge in HIV vaccine development. Phase 2b/3 vaccine trials, including the recent HVTN 706, HVTN 705, and HVTN 702 trials testing different vaccine platforms and regimens to elicit cross-reactive Abs against Env, have yielded no efficacy signals. HIV-1 vaccine candidates designed to generate broadly neutralizing antibodies (bNAbs) have not attained their ultimate goals, although a germline-targeting strategy utilizing a self-assembling nanoparticle vaccine with 60 copies of gp120 engineered outer domain (eOD-GT8 60mer) was reported to stimulate precursors of VRC01-class bNAbs against the CD4-binding site (CD4bs) in nearly all vaccine recipients in a phase I trial. During HIV-1 infection, bNAbs are produced after multiple years of chronic infection only in a small subset of HIV-1-seropositive individuals. While exposure to diverse variants over years has been implicated in promoting or guiding bNAb development and maturation, other factors contributing to the generation of bNAbs are not fully understood.

This study sought to examine if anti-Env Abs that form immune complexes (ICs) could exert any modulatory effects on Ab responses against bNAb epitopes on Env. IC vaccines were utilized to suppress Ab responses to the immunogenic, strain-specific glycan hole on the BG505 SOSIP.664 trimer, but the blockage did not divert the Ab responses toward broadly reactive neutralizing epitopes. Other studies tested IC vaccines of the gp120 core cross-linked or fused with CD4i mAbs against the bridging sheet that preferentially expose the CD4bs epitope for the VRC01 bnAb lineage. Immunization with these ICs promoted the generation of Abs with similar binding footprints as the VRC01-class bNAbs. A similar study examined IC vaccines of gp120 cross-linked with mAb A32 to allosterically stabilize the chemokine receptor-binding site, but IC-induced neutralizing titers were comparable to those attained by gp120 alone. In earlier studies it was also observed the allosteric effects of CD4bs mAbs that enhanced exposure and stability of the crown region of the V3 loop on gp120, resulting in greater Ab reactivity against V3. Immunization with ICs made of gp120 and a CD4bs mAb elicited higher levels of cross-reactive Ab responses against the V3 crown, but the neutralizing activity was limited to tier 1 viruses and ineffective against most HIV-1 strains that express Env with an occluded V3 crown.

Allosteric effects were similarly observed within the V1V2 domain of Env upon binding by certain V1V2-specific mAbs. In the Env trimer, three V1V2 domains create the apical cap, and each V1V2 domain is a modular 5-strand beta-barrel that includes a V2C strand capable of adopting polymorphic structures. The V2C peptide is recognized by V2p class mAbs, such as CH58, CH59, and CAP228.3D, in alpha-helical configurations. A second class of mAbs target the V2-apex (aka V2 glycan, V2 quaternary, or V2q) that includes the prototypic bNAbs PG9, PG16, PGDM1400, CHO1, and PGT145. These V2-apex bNAbs preferentially bind Env as trimers, recognize N-glycans on the V1V2 apical surface as part of their epitopes, and require that the V2C-strand assume a beta-sheet conformation. The V2w class mAbs from rhesus macaques infected with chimeric simian-chimpanzee immunodeficiency viruses (SCIV) were recently reported and shown to target the V2 apex, but these mAbs neutralize weakly because the V2w epitopes are occluded in the native closed Env conformation. The fourth class of mAbs, V2i, target epitopes near the integrin α4β7-binding motif at the underbelly of V1V2. Interestingly, the binding of V2i mAbs 2158 and 697 was observed to augment V1V2 recognition by PG9 or CHO1, respectively, indicating the capacity of V2i mAbs to induce 02allosteric changes favoring the beta-sheet structure required for the V2 apex bNAb binding.

In a past study, immunization of mice with IC vaccines composed of monomeric gp120 proteins and V2i mAb 2158 either yielded a higher V2 Ab response with no neutralizing activities or an enhanced V3 Ab response with only tier 1 virus neutralization. The current study investigated the effect of ICs on the elicitation of neutralizing Ab responses against the V2 apex on the Env trimer. To this end, an uncleaved prefusion optimized gp140 Env trimer of CRF01_AE A244 with V3 truncation (UFO-BG.ΔV3) and A244 V1V2 on a trimeric 2J9C scaffold (V1V2-2J9C) were examined as uncomplexed proteins (UC) or in complex with mAb 2158 (IC) for immunogenicity in rabbits. A V1V2-2J9C DNA vaccine previously shown to promote the targeting of Ab responses to the V2 apex (27) was also incorporated for a co-immunization regimen. The data demonstrated that V1V2-specific Abs with distinct binding profiles were produced in the groups immunized with DNA and UFO-BG.ΔV3 UC or IC vaccines versus DNA and V1V2-2J9C UC and IC vaccines. Limited heterologous neutralizing activities were detected only in the DNA/UFO UC group. Immunization with DNA/UFO IC caused varying degrees of alterations in the induction of V1V2-binding Abs, Abs that neutralize autologous tier 1 virus via the N160-glycan-dependent V2-apex epitope, and V1V2-specific Abs that mediate phagocytosis. Thus, these data demonstrate the modulatory effects of ICs on the generation of functional Abs against HIV-1.

### 2. Materials and Methods

i. Rabbit Immunization

Rabbit immunization studies were performed in the Center for Comparative Medicine and Surgery (CCMS) in the Icahn School of Medicine at Mount Sinai (ISMMS). The ISMMS CCMS is accredited by the American Association for the Accreditation of Laboratory Animal Care International and adheres to the Guide for the Care and Use of Laboratory Animals and the U.S. Public Health Service Policy on the Humane Care and Use of Laboratory Animals. Animal care and experimentation were performed in accordance to a protocol approved by the ISMMS Institutional Animal Care and Use Committee.

Rabbits were co-immunized with V1V2-2J9C DNA plasmid with a gene gun (Particle Mediated Epidermal Delivery (PMED) device, XR-1 research model, Oxford, UK) and one of the protein immunogens (UFO-BG.ΔV3 or V1V2-2J9C) as uncomplexed vaccines (UC) or IC vaccines with V2i mAb 2158. Each rabbit received four doses of DNA and protein vaccines at 4-week intervals. Blood was collected before immunization was commenced and two weeks after each vaccination.

The plasmid 418H encoding A244 V1V2-2J9C with tPA signal sequence under HCMV promotor was used as a DNA vaccine for all four groups of rabbits. In preparation for gene gun delivery, the DNA plasmid was precipitated onto 1 μm-diameter gold beads (2 μg DNA/mg of gold), and cartridges carrying the DNA (‘bullets’) were prepared according to the manufacturer's instructions. To verify the functional expression, COS-7 cells were transfected with DNA carried by the gold beads. Briefly, COS-7 cells (2×10⁵ cells/chamber) were seeded in two-chamber culture slides (Thermo Scientific Nunc) and transfected the next day by a gene gun at a pressure of 125 lb/in². Cells were incubated at 37° C. for 2-3 days and were fixed and labeled with 6 μg/ml mAb (2158 or PG9) and a 1:100 dilution of fluorescein isothiocyanate (FITC)-conjugated goat anti-human IgG (Zymax, Invitrogen). The presence of transfected cells was revealed by FITC staining visualized by fluorescence microscopy.

The UFO-BG.ΔV3 gp140 protein was constructed with an A244 gp120 subunit, a gp41 ectodomain of BG505, and an optimized helical region 1 to form an uncleaved chimeric Env trimer. The V3 crown (TIGPGQV; SEQ ID NO:10) was deleted and replaced with GR, and two amino acids were substituted (R319Y, T320F according to HXB2 location). The goal of this modification was to eliminate major V3 crown immunogenic epitopes while minimizing impact on closed trimer stability. Previous studies of the mutation effects on neutralization sensitivity indicated that the V3 crown could be released from its pocket on the trimer without overall trimer opening, indicating that a well-designed truncation may achieve this goal. Models of the V3 loop with varying crown segments (starting at positions 306-309 and ending at 316-319) replaced by two-residue connector were built and reviewed (all modeling was performed in ICM-pro, Molsoft, San Diego). Models indicated that low strain beta-turn could be formed at several lengths, but extensive truncations involving 1307 and F317 left a large cavity that can disrupt the Env apex. A focus was placed on the design that replaced segment 308:316. A connector residue pair . . . GR . . . was selected on the basis of observed residue frequencies in structurally similar loops (loop preferred residue tool, also in ICM-pro). Further V3 stability optimization was attempted at buried but hydrophilic positions R319 and T320, where a computational mutation scan indicated Y and F substitutions. Notably Y319 and F320 are also found in one of the optimized SOSIP trimer structures, PDB 6NFC. Truncated versions without and with R319Y/T320F substitutions were expressed and the latter construct with a slightly better antigenic profile was chosen.

The UFO-BG proteins with or without V3 alterations were produced in ExpiCHO-S cells using the ExpiFectamine CHO Transfection Kit (Gibco), and purified by lectin affinity chromatography (GNL, Vector Laboratories) followed by gel filtration on a Superdex 200 column (GE). The V1V2-2J9C protein was produced in 293S (GnTI−/−) cells and purified by affinity chromatography. IC vaccines were prepared at an antigen/mAb ratio of 1:3 with V2i mAb 2158 and then mixed with adjuvants. UC vaccines were mixed at the same ratio with an irrelevant anti-parvovirus mAb 860. The protein vaccines were administered subcutaneously with MPLA (monophosphoryl-Lipid A, Avanti #699800P) and DDA (dimethyldioctadecylammonium bromide, Sigma #D2779) adjuvants.

ii. ELISA

ELISA was used to verify the antigenicity of immunogens used in the immunization experiments and characterize the mAb reactivity of antigens used in the multiple bead assays. In brief, antigens were coated on the ELISA wells and reacted with mAbs followed with an enzyme-coupled anti-human IgG secondary Ab. Irrelevant anti-parvovirus mAbs 1418 or 860 were included as negative controls. Colorimetric and luminescent substrates were used interchangeably, and the respective optical density (OD) or relative luminescence unit (RLU) readings were shown. To examine the allosteric effects of 2158 on PG9 binding, 2158-bound ICs were probed with biotinylated PG9, which was then detected with an alkaline phosphatase-conjugated streptavidin.

iii. Octet Bio-Layer Interferometry (BLI)

To evaluate the antigenic property of V1V2-2J9C expressed by plasmid 418H, the supernatant from 418H-transfected 293T cells was subjected to BLI analysis using an Octet Red96 instrument (ForteBio). V1V2-specific mAbs (5 μg/ml) were immobilized on anti-hIgG Fc capture (AHC) biosensors and reacted with serially diluted supernatant. Supernatant from non-transfected 293T cells was used as an assay buffer. The baseline response was established by running the loaded AHC sensors on a blank control and subtracted from the binding curves.

iv. Multiplex Bead Ab Binding Assay

To measure the relative levels of rabbit IgG reactive to different Env and V1V2 antigens, Luminex multiplex bead assays were performed with a panel of antigens described in (35). The following reagent was obtained through the NIH HIV Reagent Program, Division of AIDS, NIAID, NIH: recombinant proteins AE.A244 delta11 gp120 (ARP-12569), C.1086 gp140C (ARP-12581), C.1086 gp120 D7 (ARP-12582), M.CON-S delta11 gp120 (ARP-12576), C.1086 V1V2-tags, and AE.A244 V1V2-tags. V1V2 on 1FD6, 2J9C, or 1KNC scaffolds were produced in Jiang et al. 2016 J. Virol. 90(24):10993-1006 and Hessel et al. Cell Rep 2019; 28(4):877-95. Cyclic V2 peptides were made commercially with an N-terminal 6×Lys-Gly. Antigens were coupled to beads with the xMAP Antibody Coupling (AbC) Kit (Luminex). Rabbit IgG binding to the bead mixture was performed as described in Weiss et al. Nat Commun. 2022; 13(1):903, except that biotinylated mouse anti-rabbit IgG (Southern Biotech) was used. For PG9- and 2158-blocking assays, beads coated with the specified antigens were treated with serially diluted rabbit sera and then reacted with biotinylated PG9 or 2158.

v. Neutralization Assay

Neutralizing activity of rabbit serum was evaluated using HIV-1 pseudoviruses in TZM.bl reporter cells. Neutralization was measured as reduction of luciferase reporter gene expression after a single round of infection. This assay has been formally optimized and validated and was performed in compliance with Good Clinical Laboratory Practices, including participation in a formal proficiency testing program. Viruses were selected from the global HIV-1 reference strains. In the standard neutralization assays, serially diluted rabbit serum was incubated with pre-titrated pseudovirus for 1 hour and then added to TZM.bl cells. Neutralization titers (50% inhibitory dose (ID50)) were determined as the serum dilution at which RLUs were reduced by 50% compared to RLU in virus control wells after subtraction of background RLU in cell control wells.

For antigen-blocking neutralization assays, diluted rabbit serum was pre-treated with a fixed amount of antigen at the designated concentration, followed by neutralization assay that was performed. The mixture was incubated with pseudovirus for 1 hour and then added to TZM.bl cells. TZM-bl cells (also called JC57BL-13) were obtained from the NIH AIDS Research and Reference Reagent Program.

vi. Antibody-Dependent Cellular Phagocytosis (ADCP) Assay

The ability of V1V2-specific Abs in rabbit serum to mediate ADCP was evaluated as previously described using fluorescent NeutrAvidin beads conjugated with A244 V1V2-2J9C and THP-1 phagocytic cells (ATCC). Mean fluorescent intensity was measured and ADCP scores over serum dilutions were calculated.

vii. Data Analysis

The binding and functional activities of serially diluted Abs were calculated as areas under the titration curves (AUCs) or ID50 titers. These values were subtracted by background levels of the particular assays, and delta AUCs or ID50 values were depicted. Statistical analysis was performed using the designated statistical tests in GraphPad Prism or R software. Correlation matrices were generated using R version 4.1.0 (The R Foundation for Statistical Computing) and corrplot package.

### 3. Results

i. Combination of DNA and Protein Vaccines Used to Target Ab Response to the V1V2 Apex

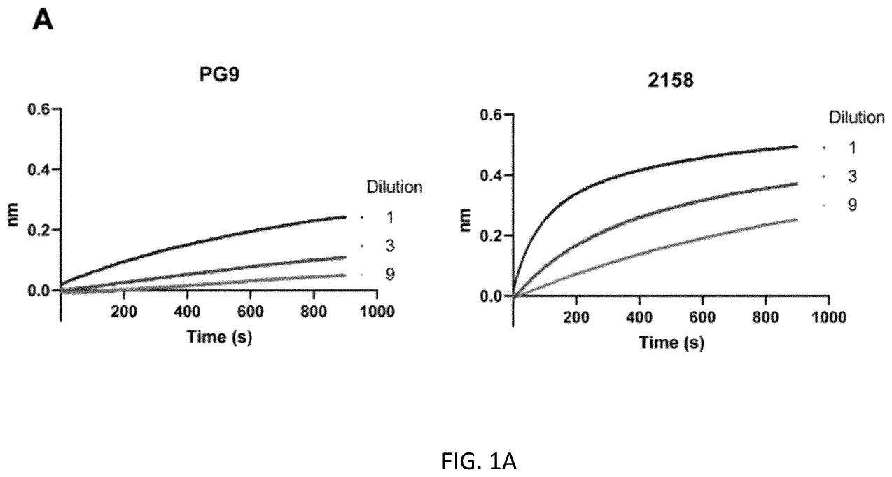

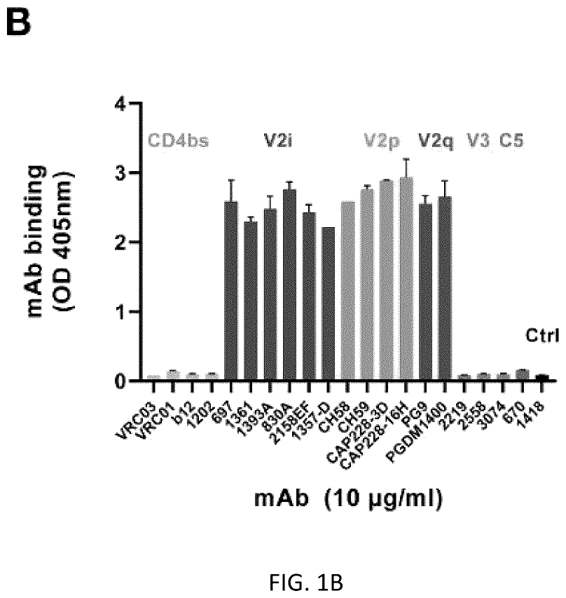

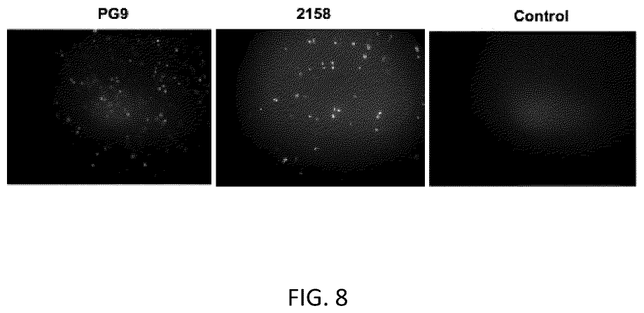

To direct Ab responses to the V1V2 apex of HIV-1 Env, a DNA+protein co-immunization strategy was employed. The V1V2-2J9C DNA vaccine expressing V1V2 of CRF01_AE A244 was delivered by a gene gun. This construct was selected because it expressed a V1V2-2J9C protein reactive with PG9 bnAb against the V2 apex (FIG. 1A left). The protein also reacted strongly with V2i mAb 2158 against the underbelly region of V1V2 (FIG. 1A right). A similar reactivity pattern was also apparent upon transfection of COS-7 cells with the DNA vaccine using gene gun (FIG. 8). ELISA analysis further showed that V1V2-2J9C was recognized by human mAbs against the three known types of V1V2 epitopes: V2i, V2p, and V2q (FIG. 1B).

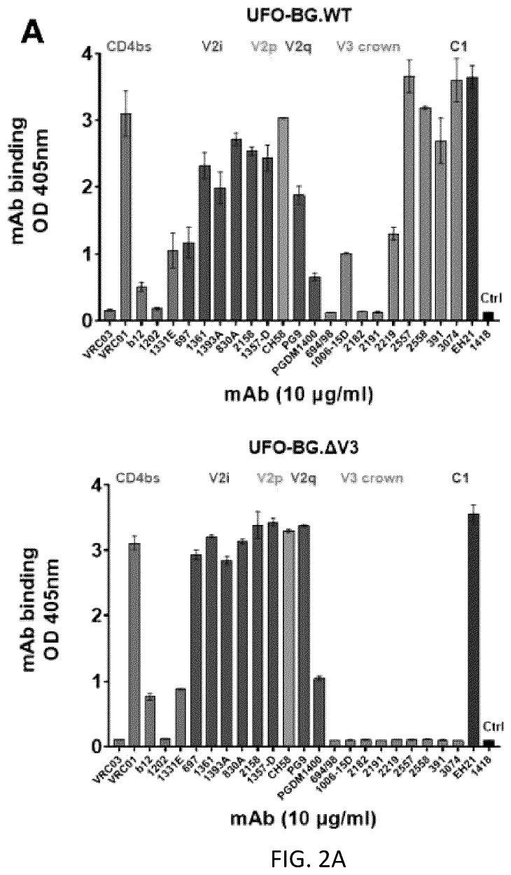

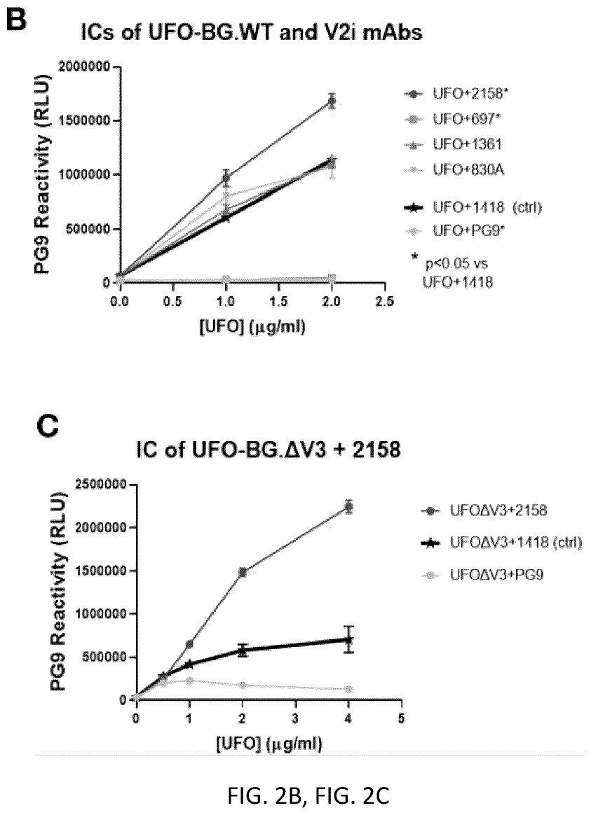

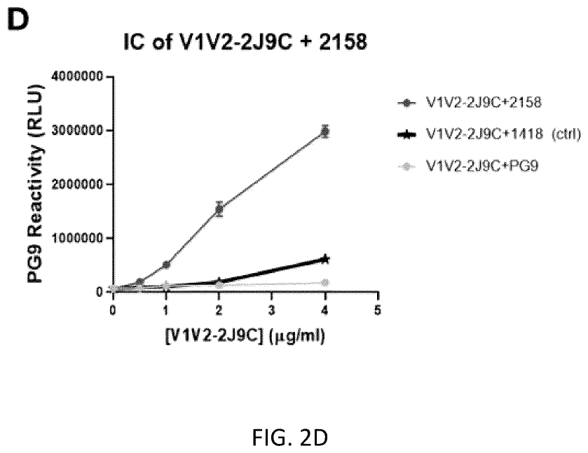

To design the protein vaccine, a trimeric uncleaved prefusion optimized (UFO) Env was produced bearing A244 gp120 with a stabilized BG505 gp41 design. The A244 UFO Env (UFO-BG.WT) was further modified by removing part of the V3 crown (UFO-BG.ΔV3) in order to direct Ab responses away from the immunogenic V3 crown epitopes. This deletion, as expected, abrogated the reactivity with V3 crown-specific mAbs, while the binding of mAbs against the different V1V2 epitopes, the CD4 binding site, and the N-terminal C1 region was comparable to that of UFO-BG.WT (FIG. 2A). These findings indicate that the Env antigenic profiles outside the V3 crown were minimally altered.

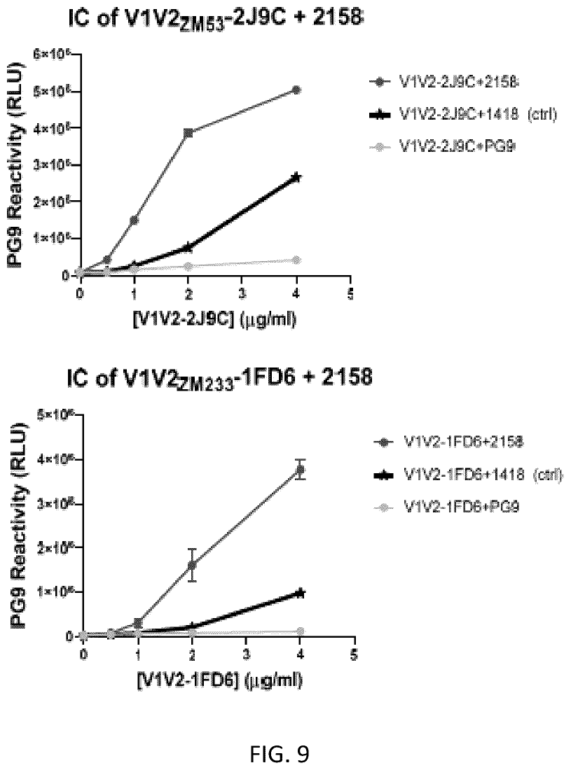

In a previous study, an IC of A244 gp120 and V2i-specific mAb 2158 was found to display enhanced reactivity with the V2 apex-specific bnAb PG9. Here it was tested whether this allosteric effect could be observed with ICs made of UFO-BG.WT and UFO-BG.ΔV3 (FIG. 2B-C). The data in FIG. 2B show a greater PG9 reactivity with 2158-bound versus uncomplexed UFO-BG.WT treated with an irrelevant mAb 1418. The enhancing effect was specific for 2158; V2i mAbs 1361 and 830A did not change PG9 binding, whereas V2i mAb 697 inhibited PG9 binding, most likely due to steric hindrance. The enhanced PG9 reactivity was similarly observed on UFO-BG.ΔV3 in complex with 2158 (FIG. 2C). This allosteric effect was also noticed when 2158 formed an IC with A244 V1V2-2J9C (FIG. 2D) and other V1V2-scaffolds (ZM53 V1V2-2J9C or ZM233 V1V2-1FD6) (FIG. 9). To target Ab responses toward the V1V2 apex, UFO-BG.ΔV3 and V1V2-2J9C of A244 were selected as protein immunogens for comparison with their respective ICs made with mAb 2158.

ii. Rabbit Immunization with DNA+Protein Combination

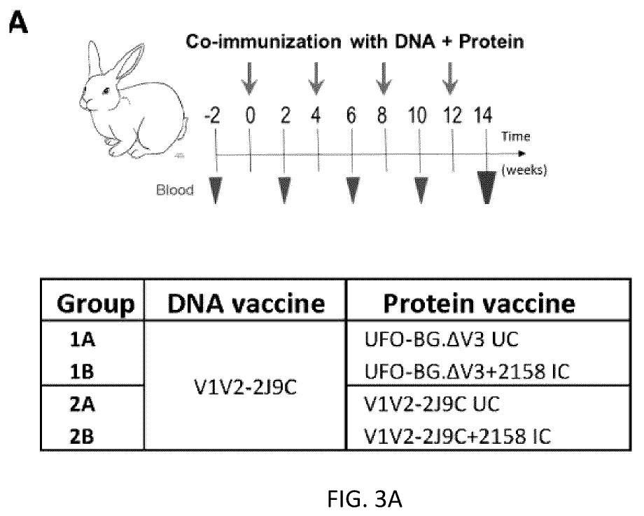

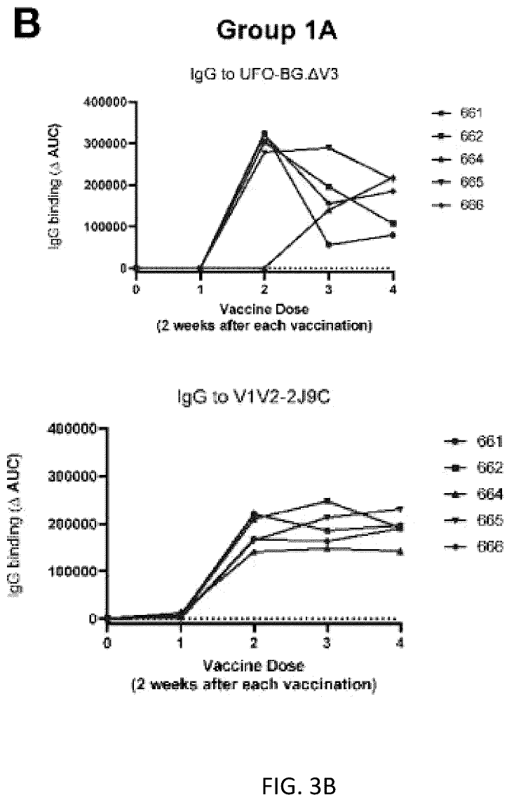

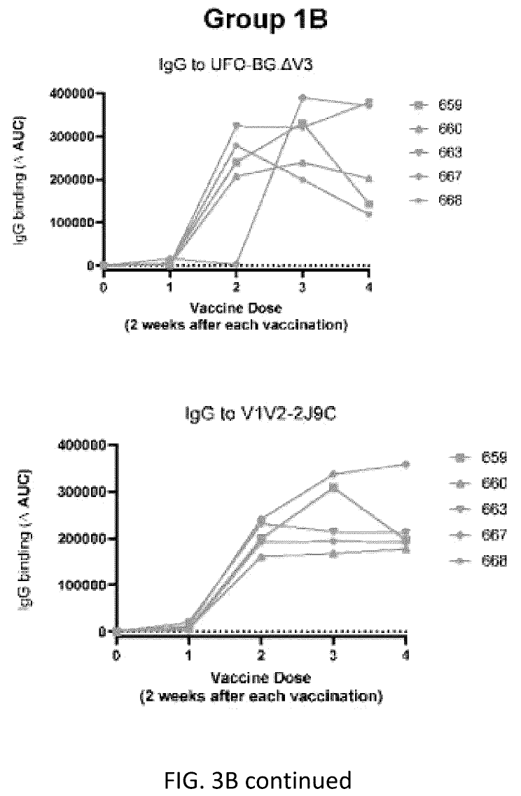

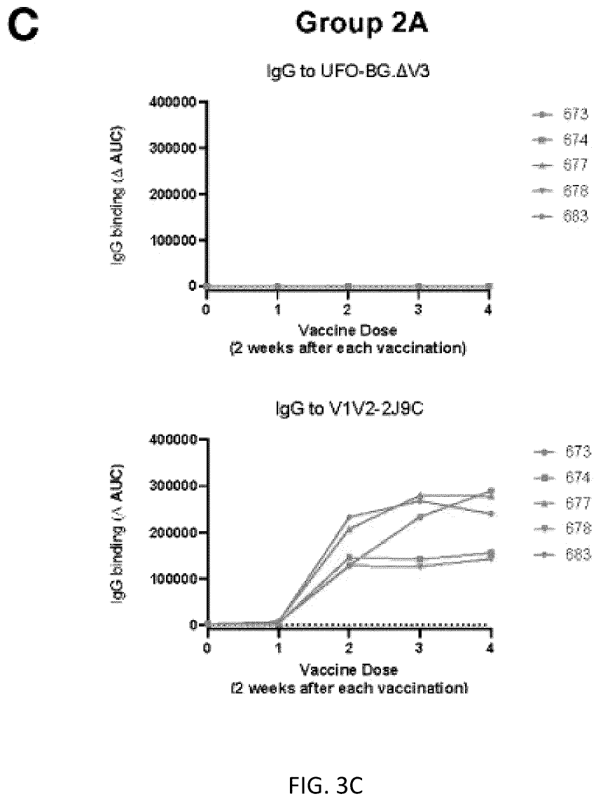

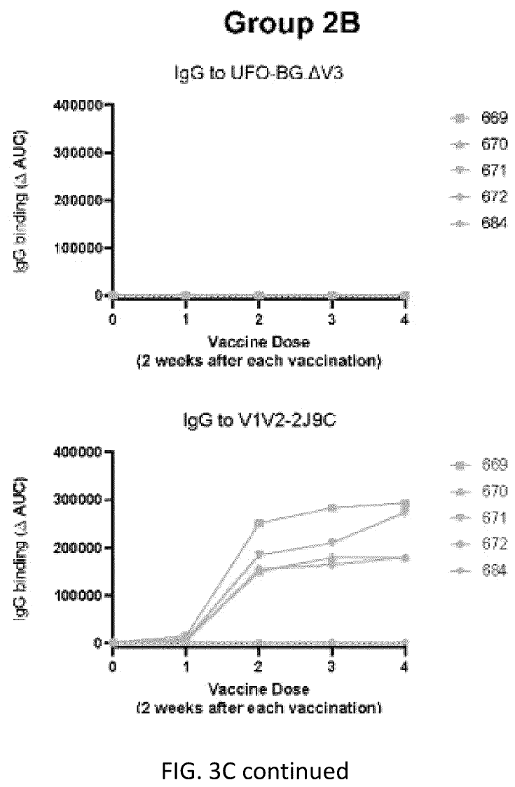

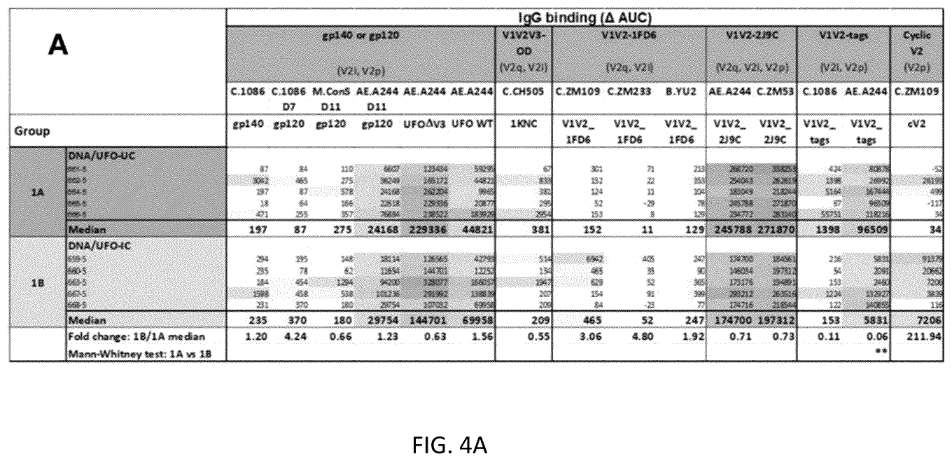

Four groups of rabbits (n=5/group) were tested for immunization with a DNA vaccine along with uncomplexed or IC protein vaccines. All rabbits were co-immunized with four doses of DNA and protein vaccines at one-month intervals. The DNA vaccine (36 .ig per dose) was delivered using a gene gun, and the protein vaccines were administered subcutaneously with MPLA (25 μg/dose) and DDA (250 μg/dose) adjuvants. For protein vaccines, antigen (100 μg/dose) and mAb (300 μg/dose) were mixed for 1 hour before addition of adjuvants. Each rabbit received the same V1V2-2J9C DNA vaccine and one of the following protein vaccines: i) uncomplexed UFO-BG.ΔV3 (Group 1A=DNA/UFO-UC), ii) UFO-BG.ΔV3±2158 IC (Group 1B=DNA/UFO-IC), iii) uncomplexed V1V2-2J9C (Group 2A=DNA/V1V2-UC), iv) V1V2-2J9C+2158 IC (Group 2B=DNA/V1V2-IC) (FIG. 3A). The uncomplexed protein vaccines were mixed with an irrelevant anti-parvovirus mAb 860. Blood was collected before immunization and 2 weeks after each vaccination to monitor the induction of serum IgG against UFO-BG.ΔV3 and V1V2-2J9C.

In Groups 1A and 1B (DNA/UFO-UC and IC), Abs against the homologous UFO-BG.ΔV3 were detected in all five rabbits after the third and fourth vaccinations (FIG. 3B). Ab responses to V1V2 were also induced after the second vaccine dose and maintained with the subsequent doses. In Groups 2A and 2B (DNA/V1V2-UC and IC), high levels of Abs were generated against the homologous V1V2-2J9C starting after two vaccinations (FIG. 3C). However, no to very low levels were detected against UFO-BG.ΔV3, indicating poor recognition of V1V2 on UFO-BG.ΔV3 by the elicited Abs.

iii. Differential Ab Responses Induced by UFO-BG.ΔV3 vs V1V2-2J9C Proteins

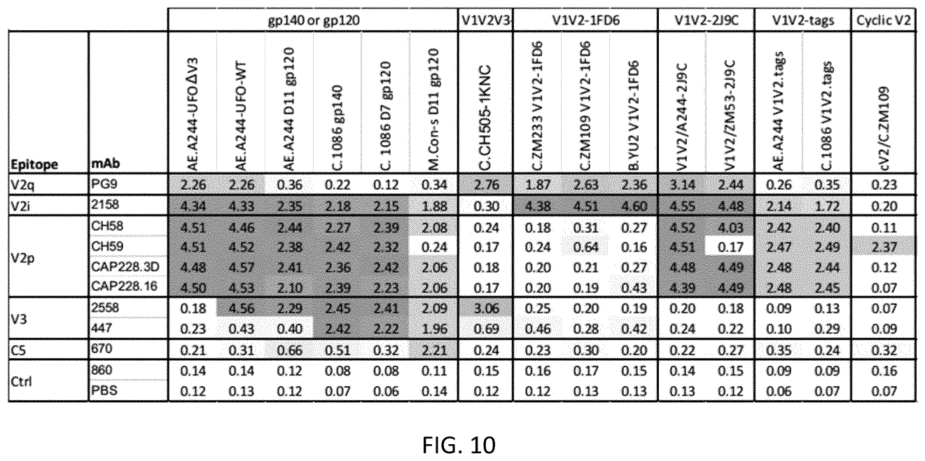

To further examine the differential Ab responses induced in the four rabbit groups, serum specimens collected two weeks after the final immunization were tested for IgG reactivity against 15 antigens, including gp140, gp120, UFO-BG, and V1V2-scaffolds from A244 and other strains, using a Luminex multiple bead assay. A panel of mAbs with defined epitope specificities was used to verify the presence or absence of distinct V2 epitopes, V3 crown, and C-terminal C5 epitope on these antigens (FIG. 10).

Groups 1A and 1B (DNA/UFO-UC and IC) displayed comparable pattern marked by robust recognition of all homologous A244 Env forms (gp120, UFO-BG.ΔV3, and UFO-BG.WT) as well as A244 V1V2 antigens (V1V2-2J9C and V1V2-tags) (FIG. 4A). Varying degrees of cross-reactivity were also observed against heterologous C.ZM53 V1V2-2J9C and C.1098 V1V2-tags, whereas Ab reactivity with other V1V2 strains on 1FD6 or 1KNC scaffolds was weak or absent, indicating a limited breadth of anti-V1V2 Abs generated in Groups 1A and 1B.

Groups 2A and 2B (DNA/V1V2-UC and IC) exhibited a starkly different pattern of Ab reactivity from Groups 1A and 1B. Both groups 2A and 2B had strong Ab reactivity with the homologous V1V2-2J9C and cross-reactivity with V1V2 of clades B and C strains on different scaffolds (C.CH505 1KNC, C.ZM109 V1V2-1FD6, C.ZM233 V1V2-1FD6, B.YU2 V1V2-1FD6, C.ZM53 V1V2-2J9C) (FIG. 4B). However, the reactivity levels with V1V2-tags were low, and no reactivity was detected against gp120, gp140 or UFO-BG Env forms, including those of the A244 strain.

iv. Minimal Changes in Binding Profiles of Abs Induced by Uncomplexed Vs. Complexed UFO-BG.ΔV3 and V1V2-2J9C

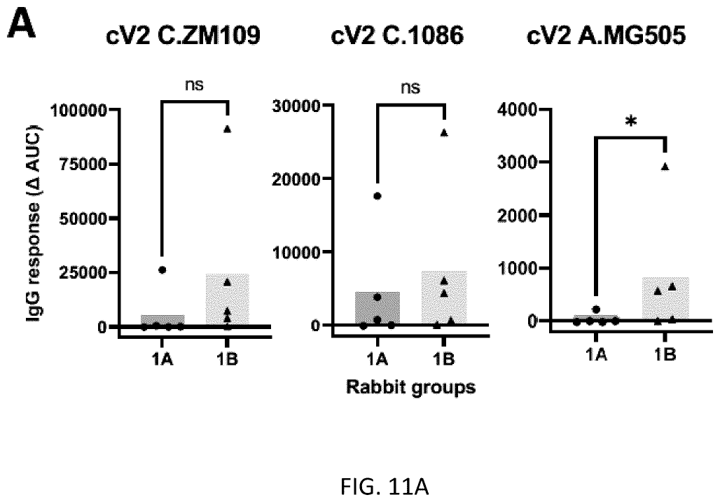

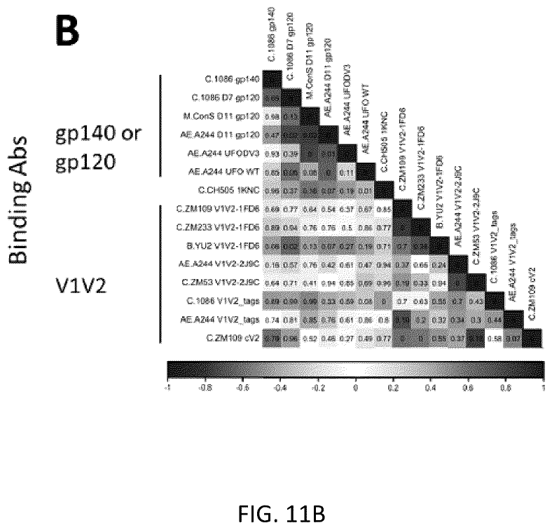

Comparisons between Groups 1A and 1B (DNA/UFO-UC vs IC) revealed that Group 1B had a lower Ab response to V1V2-tags of A244, which contains V2i and V2p epitopes, while the responses to other antigens were not significantly different (FIG. 4A, C). The response to C.ZM109 cV2 appeared to be higher in Groups 1B vs 1A. This pattern was also seen with other cV2 peptides, but a significant increase was only attained against A.MG505 cV2 (FIG. 11A). These data indicate that the UFO-BG.ΔV3 IC administered to Group 1B caused a slight skewing of Ab responses toward the V2p type epitope and away from the V2i epitope recognized by 2158. However, no increase was apparent in reactivity against V1V2-scaffolds (1KNC, 1FD6, 2J9C) bearing the V2 apex epitopes. Correlation analysis further showed weak or no positive correlation between Ab levels to Env (gp140 or gp120) and the different V1V2 antigens (FIG. 11B).

A comparison of Group 2B vs Group 2A (DNA/V1V2-IC vs UC) also showed no augmentation of the Ab responses against V1V2-scaffolds bearing the V2 apex epitopes (FIG. 4B, D). The median values against these V1V2 antigens tended to be lower in Groups 2B vs 2A. Ab responses against V1V2-tags or cV2 peptides also did not change substantially.

v. Induction of PG9- and 2158-Blocking Abs by Uncomplexed Vs Complexed UFO-BG.ΔV3 and V1V2-2J9C

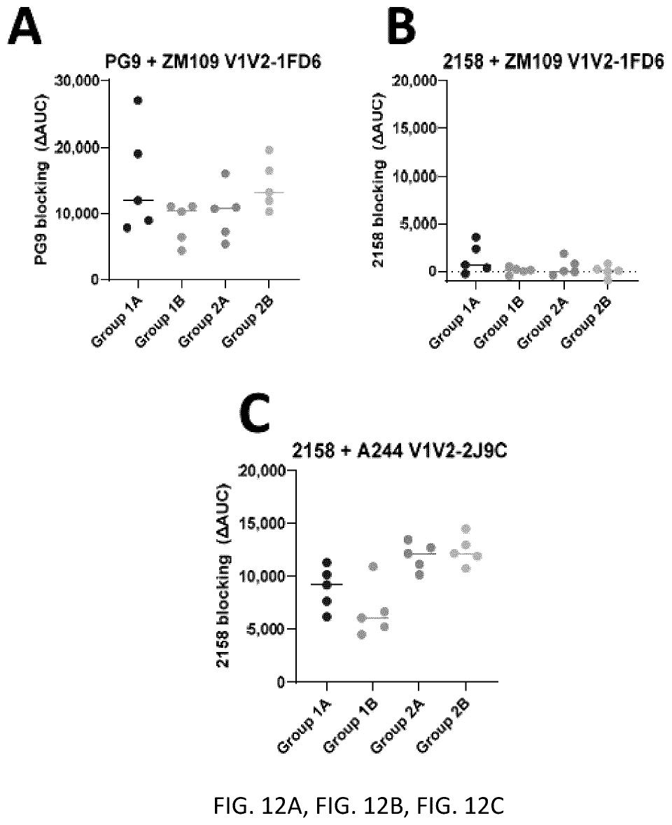

A mAb blocking assay was subsequently used to delineate the presence of serum Abs that could compete with PG9 binding to the V2q epitope. No difference was detected in the levels of PG9 blocking by sera from Groups 1B vs 1A (DNA/UFO-IC vs UC) (FIG. 12A). A trend of higher PG9 blocking was observed with sera from Groups 2B vs 2A (DNA/V1V2-IC vs UC), although a significant difference was not attained. These data indicate that the elicitation of PG9-like Abs was not improved by immunization with ICs of UFO-BG.ΔV3 and V1V2-2J9C as compared with their uncomplexed counterparts.

Immunization with 2158-containing ICs was expected to impede the induction of Abs against the V2i epitope recognized by 2158. In the blocking assay, no to low levels of Abs capable of blocking 2158 binding to C.ZM109 V1V2 on the 1FD6 scaffold were detected in all four groups of rabbits (FIG. 12B). To examine whether the V2i Abs induced in these rabbits might be strain-specific, sera to block 2158 binding to the homologous A244 V1V2 on 2J9C was tested and it was found that 2158-blocking Abs were present in sera from all four groups (FIG. 12C). The blocking levels were comparable, although they tended to be lower in Group 1B (DNA/UFO-IC). Therefore, IC vaccines containing 2158 still elicited robust Ab responses against the strain-specific V2i epitope, similar to the uncomplexed counterparts, indicating ineffective shielding of the V2i epitopes on these IC vaccines.

vi. Detection of Virus Neutralization in Rabbits Immunized with UFO-BG.ΔV3 but not with V1V2-2J9C

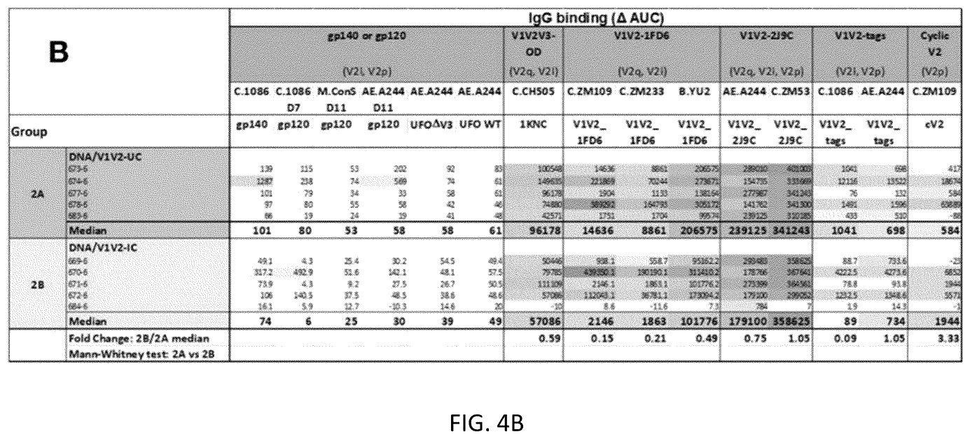

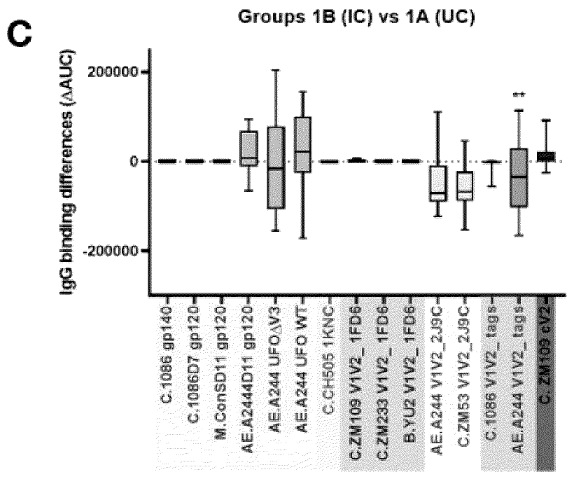

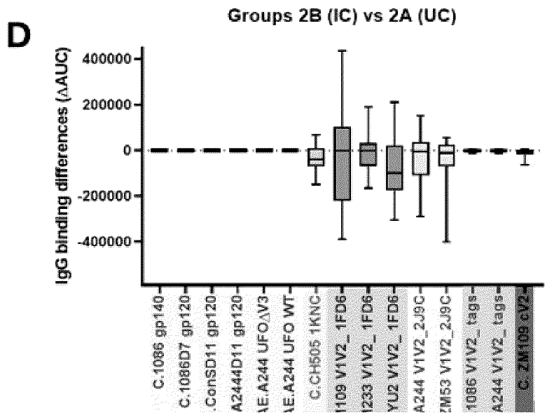

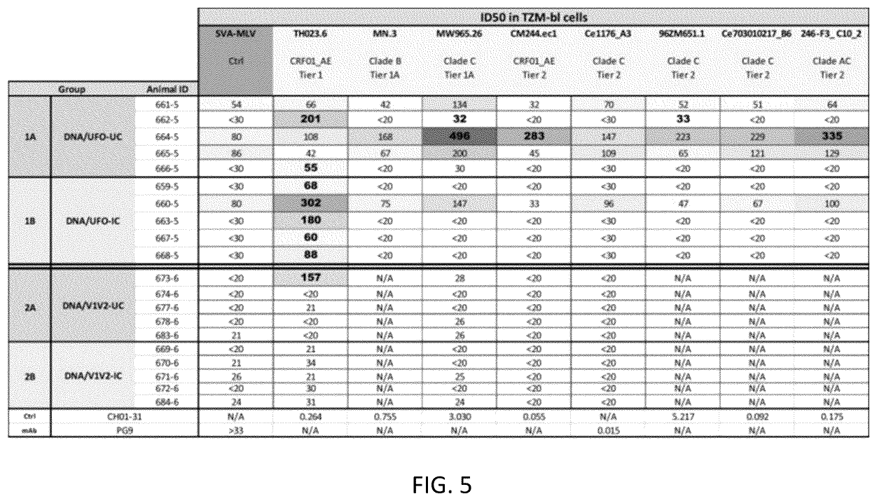

Serum neutralizing activity was assessed against HIV-1 pseudoviruses with tier 1 and tier 2 Env from clades B, C, AC, and AE. A fraction of animals in Group 1A (DNA/UFO-UC) and all in Group 1B (DNA/UFO-IC) showed neutralization against tier 1 CRF01_AE TH023.6 (FIG. 5). Group 1A (DNA/UFO-UC) also exhibited weak neutralization against few heterologous tier 1 and tier 2 viruses: two rabbits (662-5 and 664-5) showed ID50 titers above the MLV control cut-off. In Group 1B (DNA/UFO-IC), neutralization against heterologous viruses was comparable to the MLV control. These data indicate that the IC vaccine with 2158 mAb did not favor the elicitation of a heterologous neutralization response.

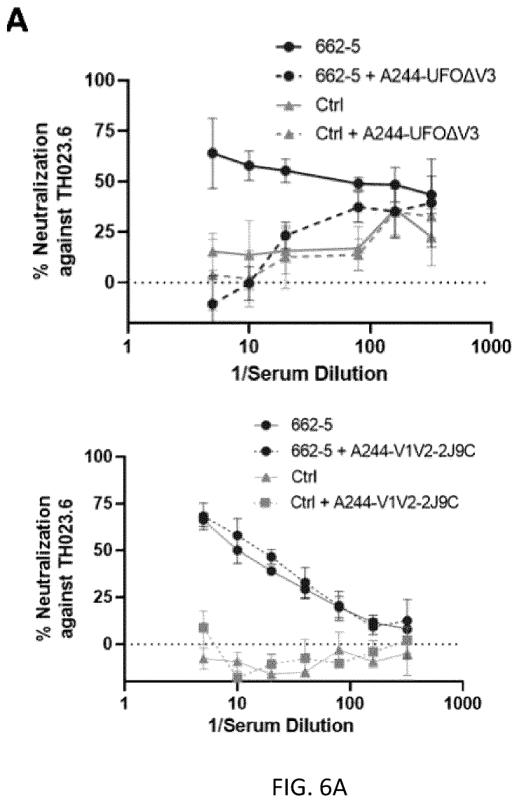

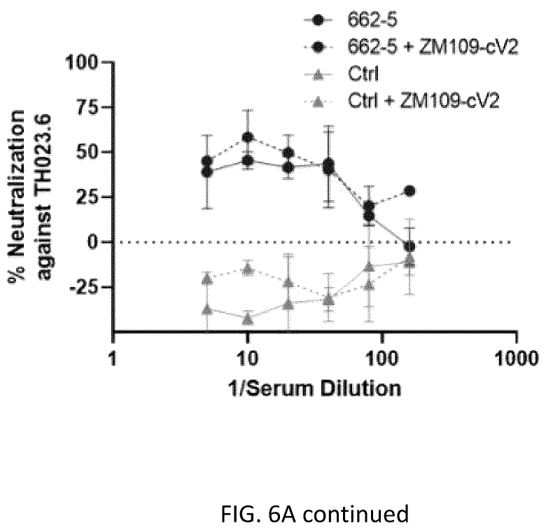

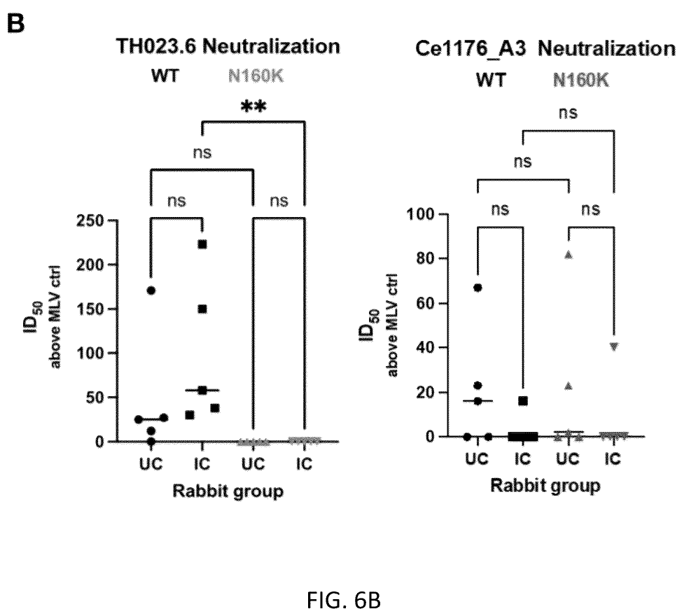

In contrast, in Groups 2A and 2B (DNA/V1V2-UC and IC) only one rabbit displayed an ID50 of 157 against TH023.6. ID50 titers of the other rabbits against all viruses tested were below the MLV control cut-off. These results indicate that the anti-V1V2 Ab responses elicited in these groups were not accompanied with virus-neutralizing activity. To define the antigenic specificity of neutralizing Abs effective against TH023.6, serum 662-5, which had the highest ID50 titer against this virus, was pre-incubated with UFO-BG.ΔV3, V1V2-2J9C, or cV2 peptide prior to testing in a neutralization assay. The data in FIG. 6A showed that TH023.6 neutralization was diminished by treatment with UFO-BG.ΔV3, but not with V1V2-2J9C or cyclic V2 antigens.

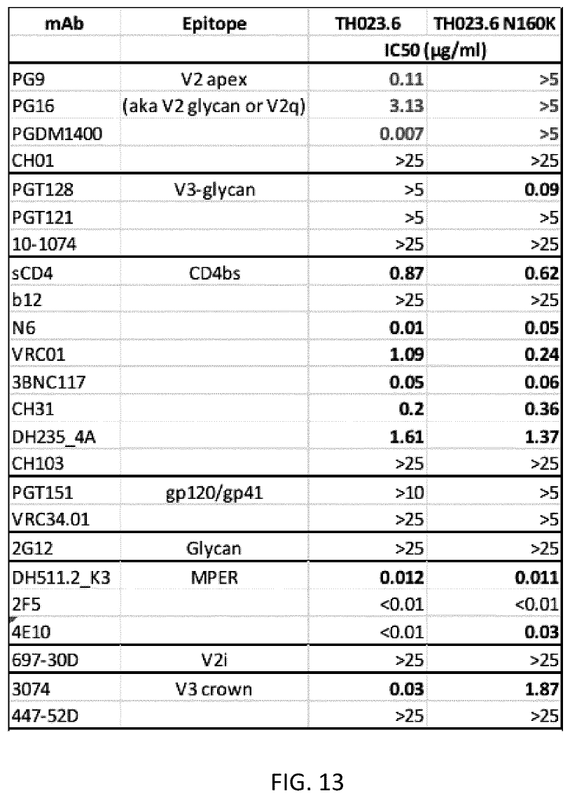

It was further evaluated whether neutralization was dependent on N160 and its glycan, which are critical for recognition by PG9 and other V2 apex bNAbs. The N160K mutation abrogated TH023.6 neutralization detected in both Groups 1A and 1B with a significant reduction noted for Group 1B (FIG. 6B). The same mutation was also tested in the context of Ce1176_A3, but neutralization against this virus was negligible and the N160K mutation had no significant effect. In line with its location in the V2 apex, N160K specifically abrogated neutralization of V2 apex-targeting bNAbs PG9, PG16, and PGDM1400, and did not reduce neutralizing potency of bNAbs against other sites, such as the CD4-binding sites, V3 glycan, or MPER (FIG. 13). This mutation also did not promote neutralization of the V3 crown- and V2i-specific mAbs, indicating that N160K did not expose these cryptic epitopes on Env. These data demonstrate that TH023.6-neutralizing Abs elicited in Groups 1A and 1B were dependent on the presence of N160 in the V2 apex, whereas neutralization against the other viruses were directed to yet undefined epitopes.

vii. Elicitation of V1V2-Specific Abs Capable of Mediating ADCP

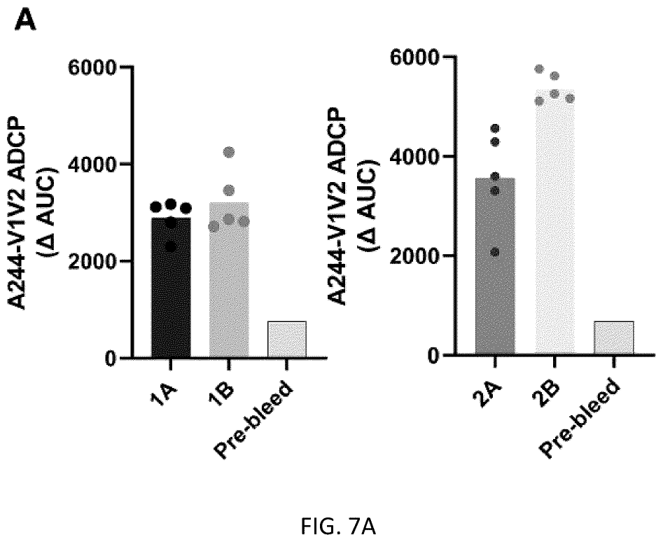

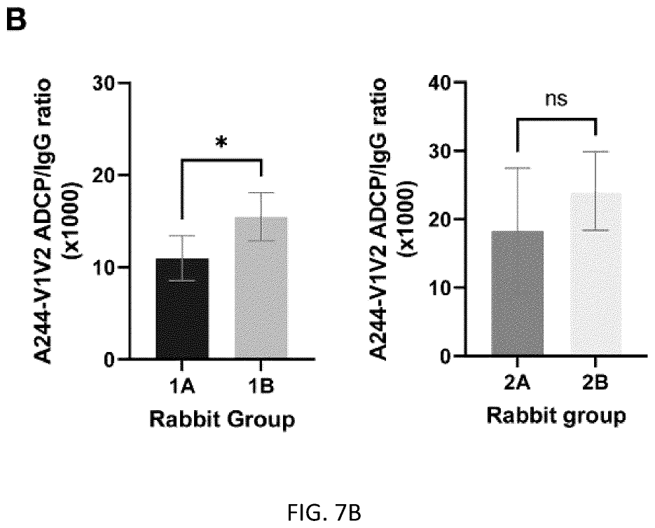

In addition to virus neutralization, Abs also mediate Fc-dependent effector functions that have been implicated in protection against HIV-1. ADCP activities of rabbit Abs have been measured in a THP-1 phagocyte assay with antigen-coated fluorescent beads. Using the same assay, V1V2-specific ADCP activities were examined in sera from all four rabbit groups. The data show that all animals in the four groups had high levels of ADCP against V1V2-2J9C of A244 (FIG. 7A). To compare the potency of Abs elicited by uncomplexed vs complexed immunogens in Groups 1A and 1B and account for differences in the serum Ab levels, the ratios of ADCP AUC scores over IgG AUC levels against the same V1V2-2J9C antigen were calculated (FIG. 7B). Group 1B showed higher ADCP potency than Group 1A, indicating a qualitative difference in the V1V2-specific Abs elicited by the UFO-BG.ΔV3 IC as compared to the uncomplexed counterpart.

Comparison of Groups 2A and 2B showed slightly higher serum ADCP levels in Group 2B, which received the V1V2-2J9C IC (FIG. 7A). However, the ratios of ADCP/IgG demonstrated comparably high ADCP potencies in both groups (FIG. 7B). Hence, both complexed and uncomplexed V1V2-2J9C induce anti-V1V2 Abs with potent effector functions.

### 4. Discussion

This study demonstrates the elicitation of neutralizing activity in sera from rabbits that received co-immunization of a V1V2-2J9C-encoding DNA vaccine and a UFO-BG.ΔV3 Env protein (Group 1A), whereas co-immunization with the same DNA vaccine and a V1V2-2J9C protein (Group 2A) was not effective. All vaccines presented the same CRF01_AE A244 sequence without the immunodominant V3 crown region. One notable rabbit in Group 1A showed heterologous neutralization against tier 1 (clade B) and tier 2 (clade AC and CRF01_AE) viruses, and another rabbit displayed homologous tier 1 neutralization (CRF01_AE) plus weak heterologous neutralization against tier 1 (clade B) and tier 2 (clade C). Hence, neutralization was attained sporadically in a fraction of animals and the titers (ID50<500) were low. Nonetheless, these data indicate the capacity of vaccines based on a single CRF01_AE A244 Env sequence to elicit neutralizing Abs across multiple clades.

Epitope mapping using N160K variants revealed that the recognition of the N160-linked glycan at the V1V2 apex, a key element common to epitopes of PG9 and other V2-apex bNAbs, was indispensable for tier 1 CRF01_AE TH023.6 neutralization which indicates the targeting of the V2 apex by the vaccine-induced neutralizing Abs. A similar finding was reported when rhesus macaques were immunized with the same V1V2-2J9C DNA vaccine along with CM244 gp145 DNA and CM244 gp120 protein vaccines. Unlike the V2-apex bNAbs, the Abs elicited against this N160-dependent site were strain specific; this was reminiscent of the V2-apex mAb 2909 that specifically neutralizes a tier 1 subtype B SF162 strain. Neutralization of heterologous viruses was mediated by Abs against yet undefined epitopes. These data indicate that co-immunization with V1V2-2J9C DNA and the UFO-BG.ΔV3 protein elicited Ab responses which conferred modest neutralization against tier 1 and limited tier 2 viruses from heterologous clades. Whether the UFO-BG V3 truncation contributes to heterologous neutralization by skewing Ab responses away from the V3 crown remains unclear and is under investigation. It should be noted, however, when a clade C C.1086 UFO design was constructed with mutations in the V2 and other Env regions without V3 crown removal, immunization of rabbits resulted in high titers of autologous neutralization especially against MW965.26, a tier 1 clade C virus extremely sensitive to Abs against V3 crown and other cryptic epitopes.

Immunization with an IC vaccine made of UFO-BG.ΔV3 and V2i mAb 2158 (Group 1B), which allosterically augments PG9 binding in vitro, resulted in a slight increase in the V2-apex-targeted neutralization of TH023.6. V1V2-specific ADCP potency was also higher in the DNA/UFO-IC group (Group 1B) as compared to the DNA/UFO-UC counterpart (Group 1A). This is in contrast with past mouse experiments in which an A244 gp120+2158 IC vaccine generated no detectable tier 1 or tier 2 neutralization and did not improve ADCP against V1V2. An IC vaccine of JRFL gp120 and 2158, on the other hand, augmented neutralization against homologous clade B tier 1 virus mediated primarily by anti-V3 crown Abs. Neutralization against heterologous viruses, though only marginally detected in the DNA/UFO-UC group (Group 1A), was not observed in the DNA/UFO-IC group (Group 1B). The V2i mAb 2158 might pose steric hindrance and thus did not favor elicitation of neutralizing Abs against the heterologous viruses, but further investigation is necessary to test this idea and define the neutralizing epitopes effective against these viruses.

In contrast to neutralization, the antigen-binding IgG activities elicited by the DNA/UFO vaccines (Group 1A) displayed a more restricted breadth, mainly recognizing Env and V1V2 antigens of the homologous A244 strain. Group 1B immunized with UFO-BG.ΔV3 IC also produced a similar pattern, although varying increases in Ab levels against V2 peptides were observed in line with a past study with the A244 gp120+2158 IC vaccine. However, no significant correlation was apparent between Abs levels against V1V2 and Env antigens. Moreover, neutralization activity against the V2 apex was absorbed by UFO-BG.ΔV3, but not by V1V2-2J9C or V2 peptide. These results indicate that the neutralizing epitopes recognized by the vaccine-elicited Abs are not presented effectively by V1V2 antigens in the panel.

Co-immunization with V1V2-2J9C DNA and V1V2-2J9C protein vaccines (Group 2A) was intended to elicit Ab responses directed solely to V1V2. While high levels of cross-reactive V1V2-specific Abs were elicited, the Abs did not recognize gp120, gp140, or UFO-BG Env proteins and had no neutralizing activity against the tier 1 or tier 2 viruses tested. The use of its IC counterpart (Group 2B) did not alter the binding and neutralization profiles. The lack of Env recognition and virus neutralization indicates that the elicited Ab responses can be directed to V1V2 conformations not present in the native Env structures. The V1V2-2J9C immunogens, however, were effective at directing Ab responses to V1V2 when used in a prime-boost regimen with gp120 or gp145 vaccines or in co-immunization with UFO-BG.ΔV3 Env studied herein. A number of other V2 apex-targeting strategies have also been designed and tested for eliciting Ab responses targeted to the V2 apex. These strategies were exemplified by clade C-based UFO designs with structure-guided mutations at the V2 apex that elicited autologous neutralization in rabbits, germline targeting immunogens that activated the human V2-apex bNAb heavy-chain precursor-expressing B cells in knock-in mice, V2-apex bNAb inferred precursor-binding SOSIPs that improved exposure of the V2-apex region, and signature-based epitope targeted vaccines that broadened neutralizing Ab responses in immunized guinea pigs. Nonetheless, vaccines capable of generating the prototypic V2-apex bNAbs in wild type animals remain elusive.

This study demonstrates that co-immunization of rabbits with a V1V2-targeting DNA vaccine and UFO-BG Env immunogen lacking the immunodominant V3 crown resulted in elicitation of neutralizing Abs against some heterologous tier 1 and limited tier 2 viruses. The neutralization breadth was manifested by Abs against V2 apex and possibly other undefined sites. The data further point to the potential interference of pre-existing Abs and ICs early during infection in eliciting neutralizing Abs.

Those skilled in the art will recognize, or be able to ascertain using no more than routine experimentation, many equivalents to the specific embodiments of the method and compositions described herein. Such equivalents are intended to be encompassed by the following claims.

1. A trimeric complex comprising an uncleaved prefusion optimized gp140 env trimer.

2. The trimeric complex of claim 1, wherein the uncleaved prefusion optimized gp140 env trimer comprises three monomeric polypeptides, wherein each monomeric polypeptide comprises a gp120 subunit and a gp41 subunit.

3. The trimeric complex of claim 2, wherein each of the three monomeric polypeptides comprises an amino acid sequence of SEQ ID NO:1, SEQ ID NO:2, SEQ ID NO:3, or SEQ ID NO:4.

4. The trimeric complex of claim 2, wherein the gp120 subunit comprises a V3 truncation which replaces amino acid sequence TIGPGQV at positions 322-328 of SEQ ID NO:2 with GR compared to wild type gp120.

5. The trimeric complex of claim 2, wherein the gp120 subunit comprises a R319Y substitution, a T320F substitution, or both.

6. The trimeric complex of claim 2, wherein the gp120 subunit is from HIV-1 strain CRF01_AE A244.

7. The trimeric complex of claim 6, wherein the gp120 subunit comprises an amino acid sequence of SEQ ID NO:6 or SEQ ID NO:5.

8. The trimeric complex of claim 2, wherein the gp41 subunit is from HIV-1 strain BG505.

9. The trimeric complex of claim 8, wherein the gp41 subunit comprises the amino acid sequence
(SEQ ID NO: 7)
SAVGIGAVFLGFLGAAGSTMGAASMTLTVQARNLLSGNPDWLPDMTVWG
IKQLQARVLAVERYLRDQQLLGIWGCSGKLICCTNVPWNSSWSNRNLSE
IWDNMTWLQWDKEISNYTQIIYGLLEESQNQQEKNEQDLLALD.

10. The trimeric complex of claim 2, wherein the gp41 subunit is ectodomain gp41.

11. The trimeric complex of claim 1, wherein the trimeric complex is a vaccine.

12. A composition comprising the trimeric complex of claim 1.

13.-15. (canceled)

16. A vaccine comprising the trimeric complex of claim 1.

17.-18. (canceled)

19. A nucleic acid construct comprising a nucleic acid sequence capable of encoding a monomeric polypeptide, wherein the monomeric polypeptide comprises a gp120 subunit and a gp41 subunit.

14.-22. (canceled)

23. A composition comprising the nucleic acid construct of claim 19.

15.-26. (canceled)

27. A vaccine comprising the nucleic acid construct of claim 19.

28.-29. (canceled)

30. A method of inducing an immune response against HIV-1 in a subject comprising administering the composition of claim 12 to a subject in need thereof.

31.-35. (canceled)

36. A method of generating neutralizing antibodies to HIV-1 comprising administering the composition of claim 12 to a subject in need thereof.

37.-40. (canceled)

41. A method of treating a subject infected with HIV-1 comprising administering the composition of claim 12 to a subject in need thereof.

42.-47. (canceled)

48. A method of generating antibodies that are cross-reactive against a broad array of HIV-1 isolates comprising administering the composition of claim 12 to a subject in need thereof.

49.-51. (canceled)


SEQUENCE LISTING (10 sequences)

SEQ ID NO: 1 - protein, 659 aa, synthetic construct
MDAMKRGLCCVLLLCGAVFVSPSQEIHARFRRGARSDNLWVTVYYGVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQPCPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSIGRFYYFGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 2 - protein, 664 aa, synthetic construct
MDAMKRGLCCVLLLCGAVFVSPSQEIHARFRRGARSDNLWVTVYYGVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQPCPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSITIGPGQVFYRTGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 3 - protein, 624 aa, synthetic construct
SDNLWVTVYYGVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQPCPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSIGRFYYFGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 4 - protein, 629 aa, synthetic construct
SDNLWVTVYYGVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQPCPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSITIGPGQVFYRTGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 5 - protein, 479 aa, synthetic construct
SDNLWVTVYYGVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQPCPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSITIGPGQVFYRTGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRCKRRVVE

SEQ ID NO: 6 - protein, 474 aa, synthetic construct
SDNLWVTVYYGVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQPCPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSIGRFYYFGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRCKRRVVE

SEQ ID NO: 7 - protein, 141 aa, synthetic construct
SAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 8 - DNA, 1989 bp, synthetic construct
  source 1..1989: mol_type = other DNA
aagcttatggatgccatgaagagaggactgtgttgcgtgctgctgctctgcggcgctgtgtttgtctcccctagccaagagatccacgctagatttagaaggggagctagaagcgacaatctgtgggtgacagtgtactatggcgtgcccgtgtggaaagaggctgacaccacactgttctgtgccagcgatgccaaagctcacgagaccgaggtgcataacgtctgggctacccatgcttgcgtgcccacagatcctaaccctcaagagatcgatctggagaacgtgacagagaactttaatatgtggaagaacaacatggtggagcagatgcaagaggacgtgattagcctctgggaccagtctctgaagccttgcgtgaaactcacacccccttgtgtgacactccactgcacaaacgctaatctgaccaaggccaatctgacaaacgtgaacaatagaacaaatgtctccaatattattggaaatatcaccgatgaggtcagaaattgcagcttcaacatgacaaccgagctgagagataagaagcagaaggtgcacgccctcttctacaaactggacatcgtccccatcgaggataacaacgactccagcgagtatagactcattaactgcaacacctccgtgatcaagcagccttgccctaagatcagcttcgaccctatccccatccactattgcacccccgccggctacgctatcctcaaatgcaacgacaagaacttcaatggcaccggcccttgcaagaacgtgtccagcgtgcagtgtacccacggaatcaaacccgtggtgtccacccagctgctgctgaatggctctctggctgaggaagagatcatcattagatccgagaacctcaccaacaatgccaagaccatcatcgtgcatctgaacaaatccgtcgtgatcaactgcacaagaccctccaacaacacaaggaccagcattggaagattttactattttggcgacatcatcggcgatattagaaaggcctactgcgagattaacggcaccgagtggaataaagctctgaaacaagtgacagagaagctgaaagaacattttaacaacaagcctatcattttccaacctcccagcggcggcgatctcgaaattaccatgcaccactttaattgtaggggagagttcttctattgtaataccaccagactcttcaacaacacatgcatcgccaacggcaccatcgagggctgcaacggcaacattacactgccttgcaaaatcaagcaaatcatcaacatgtggcaaggcgccggccaagccatgtatgctccccccatttccggcaccatcaactgcgtcagcaatattaccggcattctgctgacaagggatggaggcgccaccaacaacaccaacaacgagacctttagacccggcggcggcaacattaaggacaactggaggaacgagctgtacaagtataaggtcgtgcagattgagcccctcggagtcgcccccaccagatgcaaaaggagagtcgtggaaggaggaggaggcagcggaggaggcggcagcgccgtgggaattggagccgtgtttctcggatttctgggagctgctggctccacaatgggagccgccagcatgacactgaccgtgcaagctagaaatctgctgagcggcaatcccgattggctgcccgacatgaccgtgtggggaatcaagcagctgcaagctagagtgctggccgtcgaaaggtatctgagagaccagcagctgctgggaatctggggctgtagcggaaagctgatctgttgtaccaacgtgccttggaactccagctggagcaatagaaacctctccgagatctgggataacatgacatggctgcagtgggacaaggagatttccaactacacacagatcatttacggactgctggaggagtcccagaatcagcaagagaaaaacgagcaagatctgctcgctctggacggatcc

SEQ ID NO: 9 - protein, 261 aa, synthetic construct
MDAMKRGLCCVLLLCGAVFVSPSQEIHARFRRGARSGSMKKVEAIIRPEKLEIVKKALSDAGYVGMTVSEVKGRGVQGGIVERYCVTLHCTNANLTKANLTNVNNRTNVSNIIGNITDEVRNCSFNMTTELRDKKQKVHALFYKLDIVPIEDNNDSSEYRLINCREYIVDLIPKVKIELVVKEEDVDNVIDIICENARTGDPGDGKIFVIPVERVVRVRTKEEGKEALLEHGLEVLFQGPGHHHHHHHHSAWSHPQFEKEF

SEQ ID NO: 10 - protein, 7 aa, synthetic construct
TIGPGQV